SEQUENCE LISTING (7 sequences)

SEQ ID NO: 1 - DNA, 20 bp, Artificial sequence
  Designed primer for replication
actcactatagggaggaaga

SEQ ID NO: 2 - DNA, 35 bp, Artificial sequence
  Synthesized template strand for replication
ttctctntcttcctccctatagtgagtcgtattat

SEQ ID NO: 3 - DNA, 35 bp, Artificial sequence
  Synthesized template strand for replication
agctctntcttcctccctatagtgagtcgtattat

SEQ ID NO: 4 - DNA, 23 bp, Artificial sequence
  Designed primer for replication
ataatacgactcactatagggag

SEQ ID NO: 5 - DNA, 35 bp, Artificial sequence
  Synthesized template strand for replication
ttctcnntcttcctccctatagtgagtcgtattat

SEQ ID NO: 6 - DNA, 21 bp, Artificial sequence
  Designed primer for transcription
ataatacgactcactataggg

SEQ ID NO: 7 - RNA, 17 nt, Artificial sequence
  Product by transcription
gggaggaaganngagaa

(19) United States
(12) Patent Application Publication
(10) Pub. No.: US 2011/0087015 A1
(43) Pub. Date: Apr. 14, 2011

(54) Nucleoside and nucleotide having an unnatural base and use thereof

(75) Inventors: Ichiro Hirano, Yokohama-shi (JP); Shigeyuki Yokoyama, Yokohama-shi (JP)

(73) Assignee: RIKEN, Wako-shi (JP)

(21) Appl. No.: 10/571,138

(22) PCT Filed: Sep. 10, 2004
(86) PCT No.: PCT/JP04/13216

§ 371 Date: Jan. 5, 2007

(30) Foreign Application Priority Data
Sep. 10, 2003 (JP) 2003-318801

(51) Int. Cl. C07H 21/02; C07H 19/16; C07H 19/20; C07H 21/04

(52) U.S. Cl. 536/24.5; 536/27.21; 536/26.7; 536/26.26; 536/23.1

(57) ABSTRACT

The object of the present invention is to provide a nucleoside or a nucleotide, or a derivative thereof, which has an unnatural base. The nucleoside and others of the present invention are characterized by having a 2-amino-6-(2-thiazolyl)purin-9-yl group or a 2-amino-6-(2-oxazolyl)purin-9-yl group as a base, wherein the 4- and/or 5-position of the thiazolyl or oxazolyl group may be substituted.

### TECHNICAL FIELD

The present invention claims priority to Japanese Patent Application No. 2003-318801 filed on Sep. 10, 2003.

The present invention relates to nucleosides or nucleotides having unnatural bases and uses thereof.

### BACKGROUND ART

In nucleic acids (DNA, RNA) which are biological macromolecules, enormous amounts of genetic information essential for vital activities are recorded as sequences composed of combinations of only 4 different bases. Such a nucleic acid allows self-replication using itself as a template by the action of DNA polymerase, and further undergoes processes of RNA polymerase-mediated transcription and ribosome-mediated translation to ensure the transmission of genetic information from DNA to DNA, from DNA to RNA, and/or from RNA to protein. It is exclusive base-pairing rules (A:T/U, G:C) that enable these replication and transmission events of genetic information. In addition, nucleic acids can form a variety of higher-order structures and hence exert various functions. By way of example, it is one of the indications that a large number of novel nucleic acids having aptamer and/or ribozyme functions have been generated by in vitro selection techniques.

However, unlike proteins which are composed of 20 types of amino acids, the chemical and physical diversity of nucleic acids is limited by the fact that there are only 4 bases (2 base pairs) in natural nucleic acids. For example, functional RNAs (e.g., tRNA, rRNA, mRNA) found in living bodies utilize various modified bases to stabilize their own structure and/or RNA-RNA and RNA-protein interactions. Thus, it will be very advantageous to expand the repertory of new bases (base pairs) in developing novel functional nucleic acids.

With the aim of further expansion of nucleic acid functions, attempts have been made to design nucleosides or nucleotides having unnatural bases. There are two possible approaches for introducing modified bases (or unnatural bases) into nucleic acids: 1) direct introduction by chemical synthesis; and 2) introduction catalyzed by nucleic acid polymerase enzymes. In the case of 1), there is a need to solve some problems associated with chemical synthesis, such as the stability of amidite units and the presence of protecting groups appropriate for base moieties. If these problems are solved, various unnatural bases can be introduced in a site-selective manner. However, the nucleic acids thus obtained are difficult to amplify and it is also difficult to synthesize long-chain nucleic acids. In the case of 2), if the enzymes recognize substrates to cause replication and transcription between artificial base pairs in a complementary manner, nucleic acids containing such artificial base pairs can be amplified and prepared. However, such substrates and base pairs (unnatural nucleotides) are still under development.

If new artificial bases can be introduced through transcription into RNA in a site-specific manner, it will be possible not only to develop novel functional nucleic acids, but also to prepare artificial proteins by incorporating unnatural amino acids into proteins through genetic codes expanded due to artificial bases. The inventors of the present invention have conducted studies to develop base pairs that have hydrogen-bonding patterns different from those of natural base pairing and that are capable of eliminating base pairing with natural bases by steric hindrance; they have developed various artificial base pairs. In particular, the inventors have designed purine derivatives having a bulky substituent at the 6-position, i.e., 2-amino-6-dimethylaminopurine (x) and 2-amino-6-thienylpurine (s), as well as pyridin-2-one (y) having a hydrogen atom at the site complementary to the bulky substituent, and also have studied x:y and s:y base pairing by the efficiency of Klenow fragment-mediated incorporation into DNA and by the efficiency of T7 RNA polymerase-mediated incorporation into RNA.

As a result, the artificial base pair s-y designed on steric hindrance was found to show very high selectivity in transcription (FIG. 2). Using this s-y base pair, the substrate y was incorporated into RNA in a site-specific manner, opposite s in template DNA during transcription with T7 RNA polymerase. The inventors have further used this s-y base pair for expansion of genetic codes and creation of new codon-anticodon pairs corresponding to unnatural amino acids, and have succeeded in achieving in vitro synthesis of proteins containing unnatural amino acids in a site-specific manner by combining transcription of the s-y base pair with a translation system from cell extracts (FIG. 2). Moreover, the inventors have also achieved the development of new functional RNAs when iodo (a photo-crosslinkable group) or a biotin derivative capable of binding to avidin on a solid-phase carrier is attached to the 5-position of the base y and this modified substrate y is introduced through transcription into RNA (Japanese Patent Application No. 2002-208568 (Jul. 17, 2002), PCT/JP03/02342 (Feb. 28, 2003), unpublished yet).

As described above, the s-y base pair showed high selectivity in transcription. However, the transcription efficiency for incorporation of the substrate y opposite s in template DNA is reduced to around 50-60% as compared to the transcription efficiency of natural base pairing (FIG. 3). This is in part because there are two orientations for the thienyl group attached at the 6-position of s, which leads to a possibility that when C—H in the thienyl group is positioned on the side where base pairing occurs, steric hindrance is generated between C—H and y to prevent the incorporation of y (FIG. 4). If there are developed unnatural bases having not only high selectivity, but also high incorporation efficiency, functional RNAs and proteins can be provided in large amounts and these biopolymers can be used for practical purposes.

Patent Document 1: U.S. Pat. No. 5,432,272

Patent Document 2: U.S. Pat. No. 6,001,983

Patent Document 3: U.S. Pat. No. 6,037,120

Patent Document 4: International Publication No. WO01/005801

Non-patent Document 1: Piceirilli, J. A., Krauch, T., Morney, S. E. and Benner, S. A. (1990) Enzymatic incorporation of a new base pair into DNA and RNA extends the genetic alphabet. Nature, 343, 33-37.

Non-patent Document 2: Piceirilli, J. A., Moroney, S. E., and Benner, S. A. (1991) A C-nucleotide base pair: methylpseudouridine-directed incorporation of formycin triphosphate into RNA catalyzed by T7 RNA polymerase. Biochemistry, 30, 10350-10356.

Non-patent Document 3: Switzer, C. Y., Morney, S. E. and Benner, S A. (1993) Enzymatic recognition of the base pair between isocytidine and isoguanosine. Biochemistry, 32, 10489-10496.

Non-patent Document 4: Morales, J. C. and Kool, E. T. (1999) Minor groove interactions between polymerase and DNA: More essential to replication than Watson-Crick hydrogen bonds? J. Am. Chem. Soc., 121, 2323-2324.

Non-patent Document 5: Nagatsugi, F., Uemura, K., Nakashima, S., Maeda, M., and Sasaki, S., Tetrahedron, 53, 3035-3044, 1997

Non-patent Document 6: Wu, Y., Ogawa, A. X., Berger, M., MeMinn, D. L., Schultz, P. G. and Romesberg, F. E. (2000) Efforts toward expansion of the genetic alphabet: Optimization of interbase hydrophobic interactions. J. Am. Chem. Soc., 122, 7621-7632.

Non-patent Document 7: Tae, E. L., Wu, Y., Xia, G., Schultz, P. G. and Romesberg, F. E. (2001) Efforts toward expansion of the genetic alphabet: Replication of RNA with three base pairs. J. Am. Chem. Soc., 123, 7439-7440.

Non-patent Document 8: Ishikawa, M., Hirao, I. and Yokoyama, S. (2000) Synthesis of 3-(2-deoxy-β-D-ribofuranosyl)pyridine-2-one and 2-amino-6-(N,N-dimethylamino)-9-(2-deoxy-β-D-ribofuranosyl)purine derivatives for an unnatural base pair. Tetrahedron Letters, 41, 3931-3934.

Non-patent Document 9: Hirao, I., Ohtsuki, T., Fujiwara, T., Mitsui, T., Yokogawa, T., Okuni, T., Nakayama, H., Takio, K., Yabuki, T., Kigawa, T., Kodama, K., Yokogawa, T., Nishikawa, K., and Yokoyama, S. (2002) An unnatural base pair for incorporating amino acid analogs into proteins. Nature Biotechnology, 20, 177-182.

Non-patent Document 10: Fujiwara, T., Kimoto, M., Sugiyama, H., Hirao, I. and Yokoyama, S. (2001) Synthesis of 6-(2-thienyl)purine nueleoside derivatives that form unnatural base pairs with pyridin-2-one nucleosides. Bioorganic & Medicinal Chemistry Letters 11, 2221-2223.

Non-patent Document 11: Ohtsuki, T., Kimoto, M., Ishikawa, M., Mitsui, T., Hirao, I. and Yokoyama, S. (2001) Unnatural base pairs for specific transcription. Proc. Natl. Acad. Sci. USA, 98, 4922-4925.

Non-patent Document 12: Goodman, M. F., Creighton, S., Bloom, L. B., Petruska, J. Crit. Rev. Biochem. Mol. Biol., 28, 83-126 (1993)

### DISCLOSURE OF THE INVENTION

### Problems to be Solved by the Invention

An object of the present invention is to provide a nucleoside or a nucleotide, or a derivative thereof (hereinafter also referred to as “nucleoside and others”), which has a 2-amino-6-(2-thiazolyl)purin-9-yl group or a 2-amino-6-(2-oxazolyl)purin-9-yl group as a base, wherein the 4- and/or 5-position of the thiazolyl or oxazolyl group may be substituted.

The nucleoside and others of the present invention preferably have a 2-amino-6-(2-thiazolyl)purin-9-yl group as a base, wherein the 4- and/or 5-position of the thiazolyl group may be substituted.

Another object of the present invention is to provide a nucleic acid incorporating the above nucleotide(s). In the nucleic acid of the present invention, the above nucleotide preferably forms a base pair with a nucleotide having a 5-substituted or unsubstituted-2-oxo(1H)-pyridin-3-yl group as a base.

Yet another object of the present invention is to provide a method for preparing a nucleic acid incorporating a nucleotide having a 5-substituted or unsubstituted-2-oxo(1H)-pyridin-3-yl group as a base, which comprises effecting transcription, replication or reverse transcription by using, as a template, a nucleic acid containing the nucleotide(s) of the present invention, so that the nucleotide having a 5-substituted or unsubstituted-2-oxo(1H)-pyridin-3-yl group as a base is incorporated at a site complementary to the nucleotide of the present invention.

Yet another object of the present invention is to provide a kit which comprises a nucleic acid containing the nucleotide(s) of the present invention, and a nucleotide having a 5-substituted or unsubstituted-2-oxo(1H)-pyridin-3-yl group as a base.

### Means for Solving the Problems

To solve the problems stated above, the inventors of the present invention have designed a new base 2-amino-6-(2-thiazolyl)purine (v) by replacing the thienyl group in 2-amino-6-thienylpurine (s) with a thiazolyl group (FIG. 4). As in the case of the thienyl group in s, there are two orientations for this thiazolyl group in v, but either a sulfur or nitrogen atom is positioned on the base pairing side in either orientation. Thus, the base v causes no steric hindrance during base pairing with y because it has no sterically protruding substituent such as the C—H group in the thienyl of s. Moreover, the inventors have synthesized a nucleoside derivative of this base v to study the selectivity and efficiency of v-y base pairing in replication or translation. As a result, the inventors have found that y is efficiently introduced into RNA during transcription when v-containing template DNA is used, and have arrived as a result at the present invention (FIG. 3).

Nucleosides, nucleotides or derivatives thereof having a 2-amino-6-(2-thiazolyl)purin-9-yl group or a 2-amino-6-(2-oxazolyl)purin-9-yl group as a base

The present invention provides a nucleoside or a nucleotide, or a derivative thereof, which has a 2-amino-6-(2-thiazolyl)purin-9-yl group or a 2-amino-6-(2-oxazolyl)purin-9-yl group as a base. The 4- and/or 5-position of the thiazolyl or oxazolyl group in the base may be substituted. The nucleoside and others of the present invention typically have the structure shown in FIG. 1. Although there are two orientations for the thiazolyl or oxazolyl group in the base, the nucleoside and others of the present invention are advantageous in that in either orientation, the base causes no steric hindrance during base pairing with y because it has no sterically protruding substituent such as the C—H group in the thienyl of s.

As used herein, the term “nucleoside” is intended to mean a glycoside compound formed through glycosidic linking between a nucleic acid base and a reducing group of a sugar. It should be noted that the term “nucleic acid base” is intended to encompass adenine, guanine, cytosine, thymine, uracil, and also derivatives thereof. The type of a “derivative” of the above base is not limited in any way. Specific examples include bases equivalent to a 2-amino-6-(2-thiazolyl)purin-9-yl group and bases equivalent to a 2-amino-6-(2-oxazolyl)purin-9-yl group. The term “nucleotide” refers to a compound in which the sugar moiety of the above nucleoside forms an ester with phosphoric acid, more preferably a mono-, di- or tri-phosphate. The sugar moiety of such a nucleoside or nucleotide may be ribofuranosyl, 2-deoxyribofuranosyl, or 2-substituted ribofuranosyl having a substituent (e.g., halogen) at the 2-position. Likewise, the phosphoric acid moiety may be thiophosphoric acid. Namely, the sugar and phosphoric acid moieties may be in the same form as found in known nucleosides, nucleotides, or derivatives thereof. A ribonucleotide whose sugar moiety is ribofuranosyl can be used as a member constituting RNA, while a deoxyribonucleotide whose sugar moiety is deoxyribofuranosyl can be used as a member constituting DNA.

The nucleoside and others of the present invention typically have such a structure as shown in FIG. 1. In the base, the 4- and/or 5-position of the thiazolyl or oxazolyl group (R¹ and/or R² in FIG. 1) may be hydrogen or may be substituted with a substituent selected from the group consisting of the following:

1) a lower alkyl group;

2) a photoreactive group selected from iodine and bromine;

3) an alkenyl group, an alkynyl group or an amino group, or a derivative thereof;

4) biotin or a derivative thereof; and

5) a fluorescent molecule selected from fluorescein, 6-carboxyfluorescein, tetramethyl-6-carboxyrhodamine, and derivatives thereof. In a preferred embodiment, only one of the 4- and 5-positions is substituted. A preferred substituent is a lower alkyl group.

1) A lower alkyl group refers to a linear or branched C₁-C₄ alkyl group, including cases where two alkyl groups may together form a ring. Preferred is a methyl group.

2) A photoreactive group selected from iodine and bromine will generate radicals upon light irradiation and produce covalent bonding between adjacent molecules. This enables the formation of multimers between nucleic acids containing the nucleotide(s) of the present invention and other molecules (preferably biological molecules).

3) The substituent may also be an alkenyl group, an alkynyl group or an amino group, or a derivative thereof. These alkenyl, alkynyl and amino groups, as well as derivatives thereof are helpful in hydrophobic or hydrophilic interaction with other molecules, for example, to enhance interaction between aptamers and their target molecules. In the case of ribozymes, these groups are also helpful to create a new active site. Further, a derivative of an amino group can be used as a synthetic intermediate to prepare a derivative labeled with biotin or a fluorescent dye.

The alkenyl or alkynyl group preferably contains 2 to 5 carbon atoms, and more preferably 2 to 3 carbon atoms. Examples of their derivatives include —C≡CC₆H₅, —C≡CCH₂NH₂ and —CH═CH—CH₂—NH₂. Preferred is —C≡CC₆H₅ (a 2-phenylethynyl group).

4) Biotin is also called Coenzyme R and is a member of vitamins B. Biotin is known to specifically bind to and form a conjugate with avidin (a glycoprotein contained in albumen). Thus, a nucleoside and others having biotin as a substituent will specifically bind to avidin protein. This means that a nucleic acid containing a biotin-labeled nucleoside and others can be attached to and hence immobilized on avidin-bound carriers. If nucleic acids (e.g., aptamers) binding to specific molecules are immobilized, such immobilized nucleic acids can be used for detection and isolation of specific substances or used as diagnostic reagents, by way of example. To introduce biotin as a substituent on the nucleoside and others of the present invention, biotin may be introduced directly, but preferably via a linker selected from an aminoalkyl group, an aminoalkenyl group, an aminoalkynyl group, etc. As used herein, the term “biotin derivative” is intended to also include biotin modified to have a linker for introduction into nucleosides or nucleotides.

5) In a case where the substituent is a fluorescent molecule selected from fluorescein, 6-carboxyfluorescein, tetramethyl-6-carboxyrhodamine and derivatives thereof, a nucleic acid containing the nucleotide(s) of the present invention may be detected in a manner dependent on the type of fluorescent molecule. Thus, a nucleic acid containing the inventive nucleotide(s) having a fluorescent molecule can be used as a labeled nucleic acid probe to detect substances interacting with the nucleic acid. Without being limited thereto, fluorescein has an absorption peak wavelength of 513 nm and a fluorescence peak wavelength of 532 nm. Likewise, 6-carboxyfluorescein has an absorption peak wavelength of 495 nm and a fluorescence peak wavelength of 521 nm, while tetramethyl-6-carboxyrhodamine has an absorption peak wavelength of 555 nm and a fluorescence peak wavelength of 580 nm. Since these substances have fluorescent colors different from each other, they can also be used in multiple staining.

As used herein, unless otherwise specified, the terms “2-amino-6-(2-thiazolyl)purin-9-yl group” and “2-amino-6-(2-oxazolyl)purin-9-yl group” may include embodiments where the 4- and/or 5-position of the thiazolyl or oxazolyl group in the base is substituted.

The nucleoside and others of the present invention preferably have a 2-amino-6-(2-thiazolyl)purin-9-yl group, a 2-amino-6-(4-methyl-2-thiazolyl)purin-9-yl group or a 2-amino-6-(5-methyl-2-thiazolyl)purin-9-yl group as a base.

More specifically, the nucleoside and others of the present invention include the following:

i) 2-amino-6-(2-thiazolyl)-9-(2-deoxy-β-D-ribofuranosyl)purine;

ii) 2-amino-6-(2-thiazolyl)-9-(β-D-ribofuranosyl)purine;

iii) 2-amino-6-(2-thiazolyl)-9-(2-deoxy-(β-D-ribofuranosyl)purine phosphate;

iv) 2-amino-6-(2-thiazolyl)-9-(β-D-ribofuranosyl)purine phosphate;

v) 2-amino-6-(4-methyl-2-thiazolyl)-9-(2-deoxy-(β-D-ribofuranosyl)purine;

vi) 2-amino-6-(4-methyl-2-thiazolyl)-9-(β-D-ribofuranosyl)purine;

vii) 2-amino-6-(4-methyl-2-thiazolyl)-9-(2-deoxy-(β-D-ribofuranosyl)purine phosphate;

viii) 2-amino-6-(4-methyl-2-thiazolyl)-9-(β-D-ribofuranosyl)purine phosphate;

ix) 2-amino-6-(5-methyl-2-thiazolyl)-9-(2-deoxy-(β-D-ribofuranosyl)purine;

x) 2-amino-6-(5-methyl-2-thiazolyl)-9-(β-D-ribofuranosyl)purine;

xi) 2-amino-6-(5-methyl-2-thiazolyl)-9-(2-deoxy-β-D-ribofuranosyl)purine phosphate; and

xii) 2-amino-6-(5-methyl-2-thiazolyl)-9-(β-D-ribofuranosyl)purine phosphate. In the specification, when expressed as “2-amino-6-(2-thiazolyl),” it is also intended to include explanations on “2-amino-6-(4-methyl-2-thiazolyl)” and “2-amino-6-(5-methyl-2-thiazolyl).”

The nucleoside and others of the present invention having a 2-amino-6-(2-thiazolyl)purin-9-yl group or a 2-amino-6-(2-oxazolyl)purin-9-yl group may be synthesized in a known manner without any particular limitation. By way of non-limiting example, in Example 1 described herein later, 2-tributyltin thiazole (Compound 3a in FIG. 5) was first synthesized as a thiazole group and introduced into a known 2-amino-6-tosyloxy-9-(2-deoxy-3,5-di-O-tert-butyldimethylsilyl-(β-D-ribofuranosyl)purine (Compound 4 in FIG. 5) (Nagatsugi, F., Uemura, K., Nakashima, S., Maeda, M., and Sasaki, S., Tetrahedron, 53, 3035-3044, 1997). Subsequently, the tert-butyldimethylsilyl groups found as protecting groups on the deoxyribose group were removed to give the nucleoside of the present invention.

In another synthesis pathway for the nucleoside and others of the present invention, the tosyloxy group at the 6-position of 2-amino-6-tosyloxy-9-(2-deoxy-3,5-di-O-tert-butyldimethylsilyl-3-D-ribofuranosyl)purine (Compound 4 in FIG. 5) may be replaced by an alkylsulfonyloxy group or any other arylsulfonyloxy group. Alternatively, 2-amino-6-(2-thiazolyl)purine may be synthesized from 2-amino-6-tosyloxypurine and reacted with a deoxyribose derivative or a ribose derivative to synthesize a target compound.

The nucleoside and others of the present invention also encompass “derivatives” of the nucleoside or nucleotide. Such derivatives include, for example, a phosphoroamidite derivative and an H-phosphonate derivative.

A phosphoroamidite derivative is an embodiment where one or more substituents on a nucleoside are modified with protecting groups for use in chemical synthesis of nucleic acids (e.g., Sambrook et al., Molecular Cloning: A Laboratory Manual, the third edition, Cold Spring Harbor Laboratory, Cold Spring Harbor, N.Y. (2001), 10.42-10.46). More specifically, the 5′-hydroxyl group in a (deoxy)ribose residue may be protected with a 5′-protecting group used in nucleic acid synthesis, such as a dimethoxytrityl group (DMT), a monomethoxytrityl group or a levulinyl group. The purpose of this is to prevent the 5′-hydroxyl group from reacting with phosphoroamidite nucleosides to be charged during chemical synthesis of nucleic acids. Likewise, the trivalent phosphate group linked to the (deoxy)ribose residue on each phosphoroamidite nucleoside to be charged may be protected with a diisopropylamino group, etc. This is because the trivalent phosphate group is activated by tetrazole or the like during linking. This trivalent phosphate group may also be modified with cyanoethyl or methoxy, etc. The purpose of this is to inhibit reactions of side chains. Further, the amino group in the purine ring of the base may be protected with a phenoxyacetyl group or an isobutyryl group, etc. The purpose of this is to protect nucleophilic functions of the out-ring amino group. In the phosphoroamidite derivative of the present invention, these protecting groups are introduced at one or more positions. The protecting groups are preferably introduced at all the positions stated above. Examples of the phosphoroamidite derivative of the present invention include 2-phenoxyacetylamino-6-(2-thiazolyl)-9-[2-deoxy-5-O-dimethoxytrityl-3-O—(N,N-diisopropyl-2-cyanoethylphosphoramidyl)-β-D-ribofuranosyl]purine (Compound 9a in FIG. 6), 2-phenoxyacetylamino-6-(4-methyl-2-thiazolyl)-9-[2-deoxy-5-O-dimethoxytrityl-3-O—(N,N-diisopropyl-2-cyanoethylphosphoramidyl)-β-D-ribofuranosyl]purine (Compound 9b in FIG. 6), and 2-phenoxyacetylamino-6-(5-methyl-2-thiazolyl)-9-[2-deoxy-5-β-dimethoxytrityl-3-O—(N,N-diisopropyl-2-cyanoethylphosphoramidyl)-β-D-ribofuranosyl]purine (Compound 9c in FIG. 6).

Nucleic Acids Incorporating the Nucleotides of the Present Invention

The present invention also provides a nucleic acid incorporating one or more nucleotides having a 2-amino-6-(2-thiazolyl)purin-9-yl group or a 2-amino-6-(2-oxazolyl)purin-9-yl group as a base, wherein the 4- and/or 5-position of the thiazolyl or oxazolyl group may be substituted. The nucleic acid of the present invention encompasses single-stranded or double-stranded RNA or DNA. The double-stranded nucleic acid may be DNA/DNA, RNA/RNA, or DNA/RNA. DNA also includes cDNA obtained by reverse transcription using RNA as a template. Alternatively, the nucleic acid may form a triplex, a quadruplex, etc.

The nucleoside and others of the present invention can form a base pair with a nucleotide having a 5-substituted or unsubstituted-2-oxo(1H)-pyridin-3-yl group as a base. As illustrated in FIG. 4, the 2-amino-6-(2-thiazolyl)purin-9-yl group or the 2-amino-6-(2-oxazolyl)purin-9-yl group of the present invention forms two hydrogen bonds with 5-substituted or unsubstituted-2-oxo(1H)-pyridin-3-yl, as in the case of 2-amino-6-thienylpurine (s).

The nucleotide of the present invention which has a 2-amino-6-(2-thiazolyl)purin-9-yl group or a 2-amino-6-(2-oxazolyl)purin-9-yl group as a base, wherein the 4- and/or 5-position of the thiazolyl or oxazolyl group may be substituted, can be incorporated into nucleic acids such as DNA or RNA through transcription, replication or reverse transcription reaction. Alternatively, the nucleotide of the present invention may be incorporated into DNA or RNA through chemical synthesis, as in the case of nucleosides or nucleotides having natural bases.

These transcription, replication and reverse transcription reactions may be accomplished according to known techniques. Without being limited thereto, for example, it is possible to use T7 RNA polymerase (Takara or other suppliers) for transcription, Klenow fragment (KF) for replication, and AMV Reverse Transcriptase XL (AMV-RT, Life Science) for reverse transcription. In order to avoid removal of nucleotides having a 6-substituted 2-amino-purin-9-yl group during the reaction, the replication may also be accomplished, for example, by using Taq DNA polymerase (Takara Lae) lackin_{g} 3′→5′ exonuclease activity to effect PCR amplification of template DNA with a v-containing primer.

Although there are two orientations for the thiazolyl or oxazolyl group in the base, the nucleoside and others of the present invention are advantageous in that in either orientation, the base causes no steric hindrance during base pairing with y because it has no sterically protruding substituent such as the C—H group in the thienyl of s. For this reason, as shown in Table 1 and FIG. 9, the single-base incorporation efficiency of y opposite the nucleotide v of the present invention was Vmax/Km=1.4×10⁵, which was comparable to the incorporation efficiency between natural bases A/T. It was also about 4-fold higher than the incorporation efficiency of y opposite s. Thus, the nucleoside of the present invention has been found to achieve efficient base pairing with y. In terms of selectivity, the incorporation efficiency of y opposite v was about 3-fold higher than that of C incorporation and 20-fold or more higher than that of T incorporation.

In relation to the selective introduction of y during elongation reaction, the v-y base pair has been found to have higher replication efficiency than the s-y base pair (FIG. 12). Moreover, nucleic acids containing the nucleotides of the present invention are also useful even where two or more unnatural bases are adjacent to each other in a template. As shown in FIG. 13, when using template DNA containing two adjacent s (control), there is little incorporation of y and an elongation product is not substantially obtained. In contrast, the nucleotide v of the present invention allows replication to proceed even if two v are adjacent to each other, thereby giving a product in which two y substrates are incorporated into the complementary DNA strand.

Further, the nucleotide of the present invention is also useful in transcription reaction. More specifically; as shown in FIG. 15, the incorporation efficiency of the substrate y into RNA when using a template containing s (control) was about 50% to 60%, as compared to natural base pairing (AT). In contrast, when using a template containing v (the present invention), the incorporation efficiency of the substrate y is 96%, which is comparable to that of natural base pairing. Moreover, nucleic acids containing the nucleotides of the present invention are also useful in transcription, as in the case of replication, even if two or more unnatural bases are adjacent to each other in a template. In a case where two s (control) were adjacent to each other in a template (NN=ss), no RNA incorporating two y was obtained. In contrast, when two v were adjacent to each other in a template (NN=vv), transcription proceeded and two y substrates were incorporated into RNA although the transcription efficiency was about 30% (FIG. 16).

In this way, the use of v instead of the base s enables improvement in the incorporation efficiency of the substrate y during both replication and transcription, as expected. Moreover, the use of v also enables the preparation of conventionally unavailable DNA and RNA in which two or more unnatural y bases are located adjacent to each other. This is the first case that allows the development and mass production of novel functional RNAs and proteins, in which functional components are incorporated into RNA through artificial base pairing, and hence greatly contributes to the commercialization of these novel biopolymers.

The nucleic acid incorporating the nucleotide(s) of the present invention may be used as tRNA, mRNA, antisense DNA or RNA, a ribozyme or an aptamer. The term “antisense DNA or RNA” refers to DNA or RNA capable of inhibiting the expression of a specific gene. It was named to mean that such DNA or RNA is complementary to the full-length or partial sequence of a target gene sequence (sense strand). Antisense DNA or RNA may be used as a tool for artificial regulation of gene expression. Because of containing unnatural bases, such antisense DNA or RNA incorporating the nucleotide(s) of the present invention can be designed to have a different complementarity to a target when compared to the case of using natural bases only. The term “ribozyme” is a generic name for catalysts composed of RNA. The term “aptamer” refers to an in vitro-selected nucleic acid having the ability to bind to a specific molecule such as a protein.

DNA or RNA (e.g., mRNA, synthetic RNA) incorporating the nucleotide(s) of the present invention may also encode all or part of a protein or peptide. The nucleic acid of the present invention may be used, e.g., as a gene fragment or a probe. The present invention also encompasses the following embodiments: partial or complete replacement of native genes by the nucleic acids of the present invention; addition of one or more nucleotides of the present invention to native genes; or combinations thereof. Such non-native genes containing the nucleic acids (nucleotides) of the present invention may be modified in the same manner or according to conventional modification techniques for native genes. Thus, as in the case of conventional native genes, non-native genes containing the nucleic acids of the present invention can be expressed by insertion into appropriate expression vectors and transformation into appropriate host cells.

Moreover, it is also possible to design a new codon containing the nucleotide of the present invention. As one embodiment of the nucleotide of the present invention, an explanation will be given of a nucleotide (v) containing a base 2-amino-6-(2-thiazolyl)purine. As described above, even in a case where two or more nucleotides of the present invention are located adjacent to each other in a template, both replication and transcription reactions can proceed, so that a nucleotide (y) having a 5-substituted or unsubstituted-2-oxo(1H)-pyridin-3-yl group as a base is incorporated at a complementary site. Thus, the method of the present invention enables the preparation of conventionally unavailable DNA and RNA in which two or more unnatural y bases are located adjacent to each other. In turn, it is also possible to design a codon containing three y (yyy), those containing two y (e.g., yyA, Gyy, yGy) and those containing one y (e.g., yAG, CyT, AGy). Likewise, codons containing v can also be prepared. Such a new codon may encode either a natural amino acid or an unnatural amino acid. Further, such a new codon may encode a function including transcription or transport. In this way, the present invention not only provides novel unnatural artificial bases, but also enables the design of entirely new genetic codes by designing new codons containing the nucleotides of the present invention, thus providing a world of new genetic codes.

Further, by designing tRNA systems corresponding to the new codons of the present invention, it is also possible to design a new protein synthesis system in which numerous amino acids can be used. Amino acids which can be used are not limited in any way as long as they can be used in the protein-synthesizing enzyme system in ribosomes. Thus, the present invention provides a new protein synthesis system using the above codons of the present invention. According to the protein synthesis system of the present invention, when a nucleic acid corresponding to a codon at a desired site is efficiently replaced by the nucleic acid of the present invention or when the nucleic acid of the present invention is efficiently introduced at a desired site, it is possible to produce a protein containing a desired unnatural amino acid(s).

Furthermore, the nucleic acids of the present invention incorporating nucleotides having unnatural bases may also be used in RNA interference (RNAi). RNA interference is a phenomenon in which double-stranded RNA (dsRNA) induces mRNA degradation in a sequence-specific manner and hence inhibits gene expression. In a typical example of RNA interference, dsRNA is processed by Dicer belonging to the RNaseIII family into siRNA (short interfering RNA) of approximately 21 to 23 bases in length, which has a 3′-terminal overhang of approximately 2 bases. siRNA is associated into an siRNA-protein complex called RISC and induces mRNA degradation in a sequence-specific manner. RNA interference is shown to be a phenomenon conserved among a wide range of organism species including mammals (e.g., human, mouse), nematodes, plants, drosophila and fungi. The nucleic acids of the present invention incorporating nucleotides having unnatural bases can be used as siRNA in RNA interference or as a part of mRNA to be degraded.

Method for Preparing Nucleic Acids Incorporating Nucleotides having Unnatural Bases

The present invention further provides a method for preparing a nucleic acid incorporating a nucleotide having a 5-substituted or unsubstituted-2-oxo(1H)-pyridin-3-yl group as a base. The method of the present invention comprises effecting transcription, replication or reverse transcription by using, as a template, a nucleic acid containing the nucleotide(s) of the present invention, so that the nucleotide having a 5-substituted or unsubstituted-2-oxo(1H)-pyridin-3-yl group as a base is incorporated at a site complementary to the nucleotide of the present invention.

As described above, even in a case where two or more nucleotides of the present invention are located adjacent to each other in a template, both replication and transcription reactions can proceed, so that a nucleotide having a 5-substituted or unsubstituted-2-oxo(1H)-pyridin-3-yl group as a base is incorporated at a complementary site. Thus, the method of the present invention also enables the preparation of conventionally unavailable DNA and RNA in which two or more unnatural y bases are located adjacent to each other.

The present invention furthermore provides a kit for use in the above method. The kit of the present invention comprises a nucleic acid containing the nucleotide(s) of the present invention, and a nucleotide having a 5-substituted or unsubstituted-2-oxo(1H)-pyridin-3-yl group as a base. In the kit, the nucleic acid containing the nucleotide(s) of the present invention may be used as a template for transcription, replication or reverse transcription reaction in the method of the present invention.

### BRIEF DESCRIPTION OF THE DRAWINGS

FIG. 1 shows the structure of an embodiment of the nucleoside and nucleotide according to the present invention.

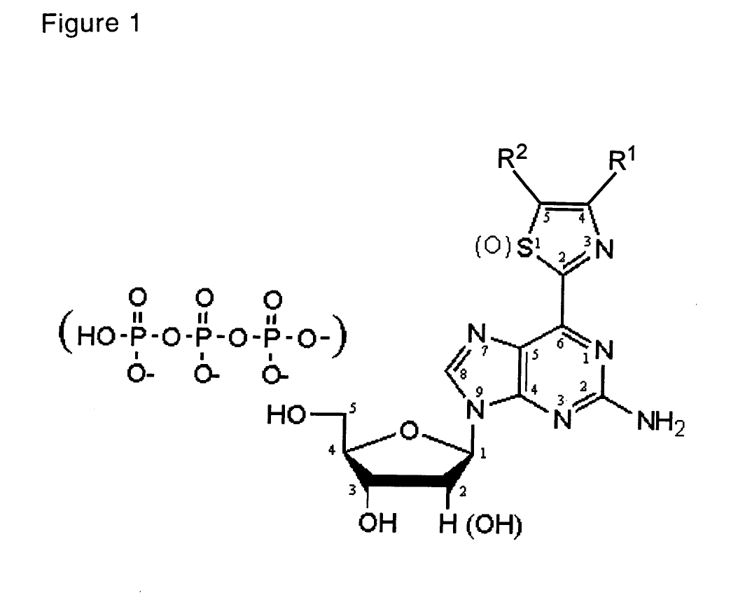

FIG. 2 shows artificial base pairing between 2-amino-6-thienylpurine (s) and pyridin-2-one (y), along with a scheme for protein synthesis using the same.

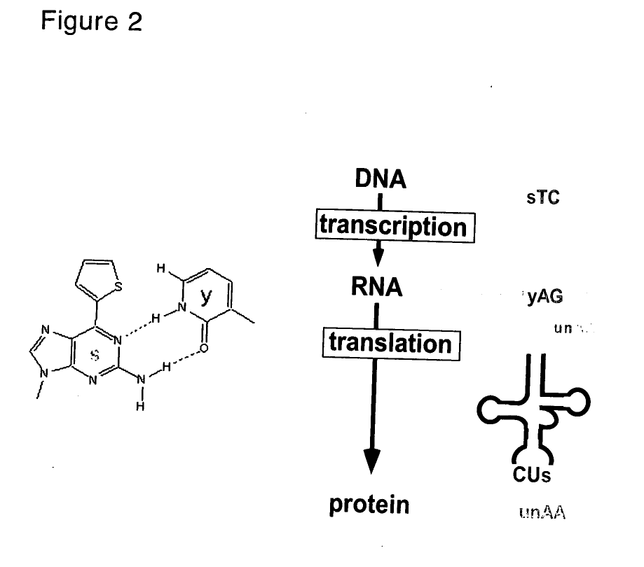

FIG. 3 shows the selectivity and efficiency of transcription reaction using artificial base pairing between 2-amino-6-thienylpurine (s) and pyridin-2-one (y) as well as artificial base pairing between 2-amino-6-(2-thiazolyl)purine (v) and y.

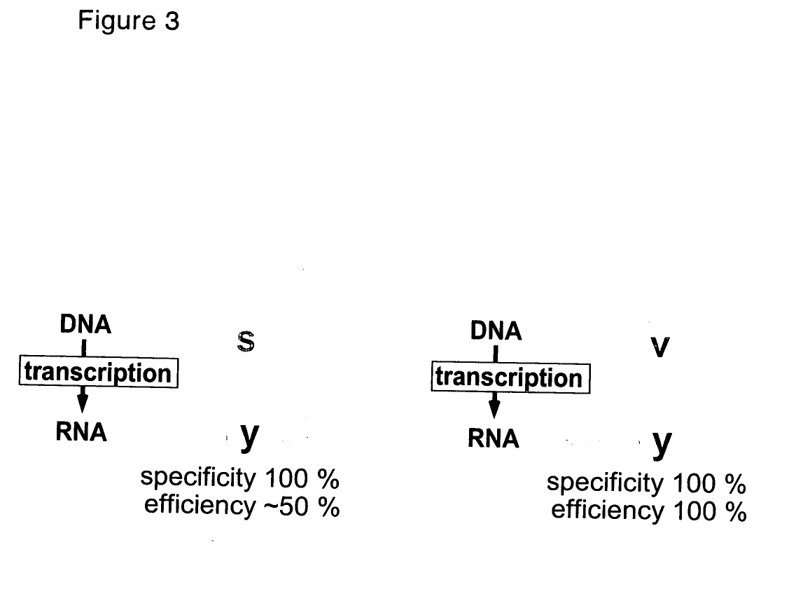

FIG. 4 shows the orientations and steric hindrance of artificial base pairing between 2-amino-6-thienylpurine (s) and pyridin-2-one (y) as well as artificial base pairing between 2-amino-6-(2-thiazolyl)purine (v) and y.

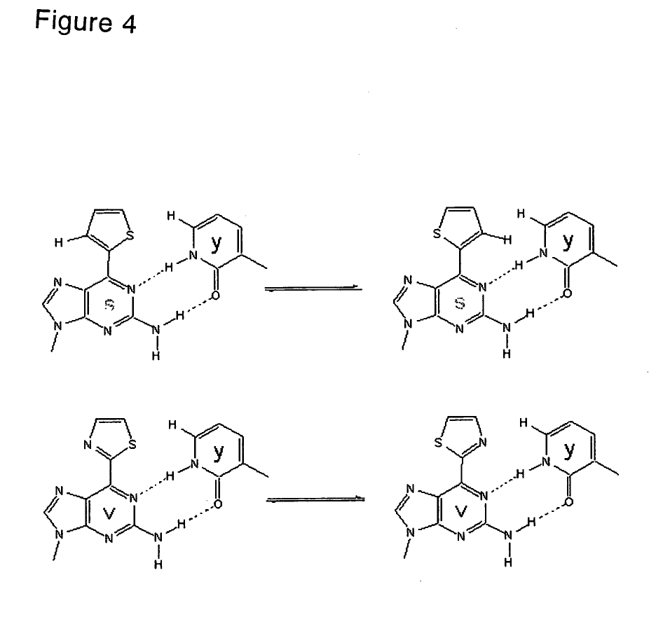

FIG. 5 shows a synthesis scheme for the nucleoside of the present invention, 2-amino-6-(2-thiazolyl)-9-(2-deoxy-β-D-ribofuranosyl)purine.

In FIG. 5, R=t-butyl-dimethylsilyl; Ts=tosyl; a series: R¹═R²═H; b series: R¹═CH₃, R²═H; c series: R¹═H, R²═CH₃.

FIG. 6 shows a synthesis scheme for the nucleoside derivative of the present invention, 2-phenoxyacetylamino-6-(2-thiazolyl)-9-[2-deoxy-5-O-dimethoxytrityl-3-O—(N,N-diisopropyl-2-cyanoethylphosphoramidyl)-β-D-ribofuranosyl]purine.

In FIG. 6, Pac=phenoxyacetyl; DMT=4,4′-dimethoxytrityl; a series: R¹═R²=H; b series: R¹═CH₃, R²═H; c series: R¹═H, R²═CH₃.

FIG. 7 shows a synthesis scheme for the nucleotide of the present invention, 2-amino-6-(2-thiazolyl)-9-(2-deoxy-(β-D-ribofuranosyl)purine 5′-triphosphate.

In FIG. 7, PPP=triphosphate; a series: R¹═R²═H; b series: R¹═CH₃, R²═H; c series: R¹═H, R²═CH₃.

FIG. 8 shows the nucleotide sequences of the primer and templates used in Klenow fragment-mediated single nucleotide insertion reaction, along with polyacrylamide electrophoretic patterns of the reaction products.

FIG. 9 shows the analysis results of the reaction rate in Klenow fragment-mediated single nucleotide insertion reaction.

FIG. 10 shows the nucleotide sequences of the primer and templates used in the reaction rate analysis of Klenow fragment-mediated single nucleotide insertion reaction.

FIG. 11 shows the nucleotide sequences of the primer and template used in Klenow fragment-mediated elongation reaction.

FIG. 12 shows polyacrylamide electrophoretic patterns of the reaction products from Klenow fragment-mediated elongation reaction.

FIG. 13 shows polyacrylamide electrophoretic patterns of the reaction products from Klenow fragment-mediated elongation reaction.

FIG. 14 shows a scheme of transcription reaction.

FIG. 15 shows polyacrylamide electrophoretic patterns of the reaction products from transcription reaction using temp35 N-1. When the transcription efficiency in Lane 5 was set to 100%, the efficiency in Lanes 1, 2, 3 and 4 was 23%, 96%, 24% and 60%, respectively.

FIG. 16 shows polyacrylamide electrophoretic patterns of the reaction products from transcription reaction using temp35 N-2. When the transcription efficiency in Lane 5 was set to 100%, the efficiency in Lanes 1, 2, 3 and 4 was 2%, 35%, 1% and 6%, respectively.

### EXAMPLES

The present invention will now be further described in the following examples, which are not intended to limit the technical scope of the invention. Based on the detailed description, various changes and modifications will be apparent to those skilled in the art, and such changes and modifications fall within the technical scope of the invention.

### Example 1

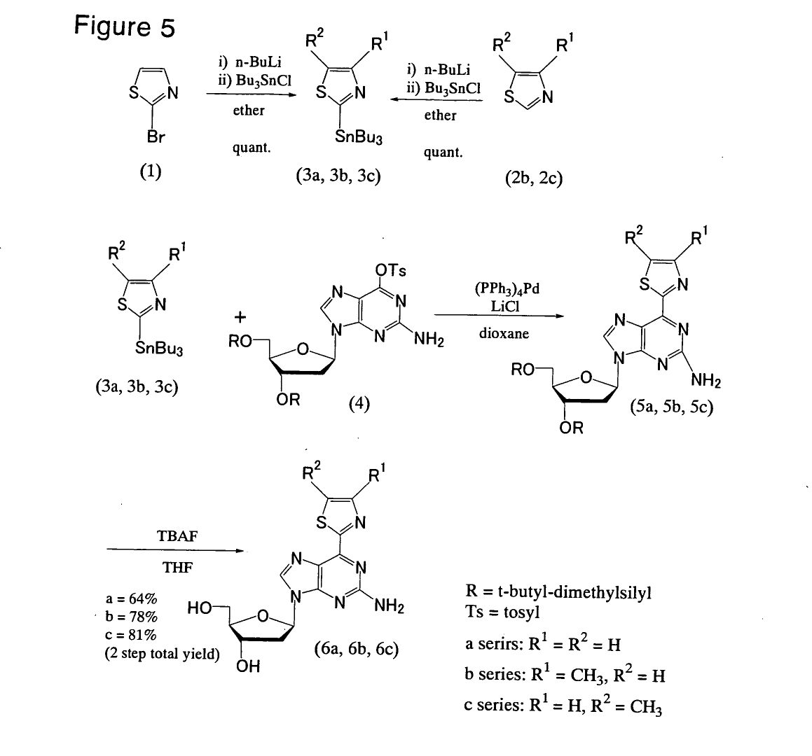

### Synthesis of a 2-amino-6-(2-thiazolyl)-9-(2-deoxy-(β-D-ribofuranosyl)purine derivative (FIGS. 5-7)

### 1) Synthesis of 2-tributyltin thiazole (Compound 3a) (FIG. 5)

Under an argon atmosphere, n-butyllithium (1.57 M in hexane, 3.2 ml, 5.0 mmol) was added to diethyl ether (25 ml) which had been cooled to −78° C. Subsequently, 2-bromothiazole (Compound 1) (450 μl, 5.0 mmol) was added dropwise at −78° C. and stirred for 30 minutes. To this solution, tributyltin chloride (1.5 ml, 5.5 mmol) was added dropwise at −78° C., and the mixture was normally warmed while stirring until its temperature reached room temperature (30 minutes).

After this reaction mixture was washed three times with saturated aqueous sodium chloride, the organic layer was dried over MgSO₄ and evaporated under reduced pressure to remove the solvent, thereby obtaining 2-tributyltin thiazole (Compound 3a) (2.1 g, yellow liquid). 2-Tributyltin thiazole thus obtained was used as such in the subsequent reaction without further purification.

### 2) Synthesis of 2-amino-6-(2-thiazolyl)-9-(2-deoxy-β-D-ribofuranosyl)purine (Compound 6a) (FIG. 5)

2-Amino-6-tosyloxy-9-(2-deoxy-3,5-di-O-tert-butyldimethylsilyl-β-D-ribofuranosyl)purine (Compound 4) was synthesized according to Nagatsugi et al. (Nagatsugi, F., Uemura, K., Nakashima, S., Maeda, M., and Sasaki, S., Tetrahedron, 53, 3035-3044, 1997). Compound 4 (490 mg, 0.75 mmol), Pd(PPh₃)₄ (44 mg, 0.04 mmol) and LiCl (64 mg, 1.5 mmol) were mixed in dioxane (9.4 ml) and bubbled with argon while stirring for 15 minutes, followed by addition of 2-tributyltin thiazole (Compound 3a) synthesized in 1) (1.4 g, 3.8 mmol). After bubbling with argon for an additional 15 minutes, the reaction mixture was refluxed on an oil bath for 3 hours. After the reaction mixture was concentrated, the residue was purified by silica gel column chromatography (eluted with 5% MeOH in CH₂Cl₂). The resulting 2-amino-6-(2-thiazolyl)-9-(2-deoxy-3,5-di-O-tert-butyldimethylsilyl-β-D-ribofuranosyl)purine (Compound 5a) (430 mg, crude product) was dissolved in THF (7.5 ml) and then, after addition of TBAF (1 M THF solution, 2.3 ml), stirred at room temperature for 15 minutes. After the reaction mixture was concentrated, the residue was purified by silica gel column chromatography (eluted with 5% MeOH in CH₂Cl₂).

The resulting product was finally purified by RP-HPLC (19×150 mm, water μ bond sphere 5μ C18 100μ, flow rate: 10 ml/min, 10%-50% CH₃CN in H₂O, 15 minutes, linear gradient) to give the desired product 2-amino-6-(2-thiazolyl)-9-(2-deoxy-β-D-ribofuranosyl)purine (Compound 6a) (155 mg, 64% yield for 2 steps, yellow solid).

¹H-NMR (270 MHz, DMSO-d₆) δ 2.26 (m, 1H), 2.65 (m, 1H), 3.55 (m, 2H), 3.84 (m, 1H), 4.38 (m, 1H), 4.96 (t, 1H, J=5.4 Hz), 5.30 (d, 1H, J=4.0 Hz), 6.29 (t, 1H, J=6.5 Hz), 6.74 (s, 2H), 8.00 (d, 1H, J=3.2 Hz), 8.12 (d, 1H, J=3.2), 8.41 (s, 1H);

¹³C-NMR (68 MHz, DMSO-d₆) δ 39.32, 61.55, 70.58, 82.52, 87.57, 122.66, 123.93, 141.68, 144.66, 147.36, 154.78, 159.68, 164.03;

HRMS (FAB, 3-NBA matrix) calculated for C₁₃H₁₅N₆O₃S (M+1): 335.0926, found: 335.0922; UV-vis (in EtOH) λmax=360 nm (ε=8030), 298 nm (ε=8620), 231 nm (ε=18080), λmin=326 nm (ε=4240), 265 nm (ε=3450), 215 nm (ε=9660); TLC Rf=0.12 (CH₂Cl₂:MeOH=9:1, v/v).

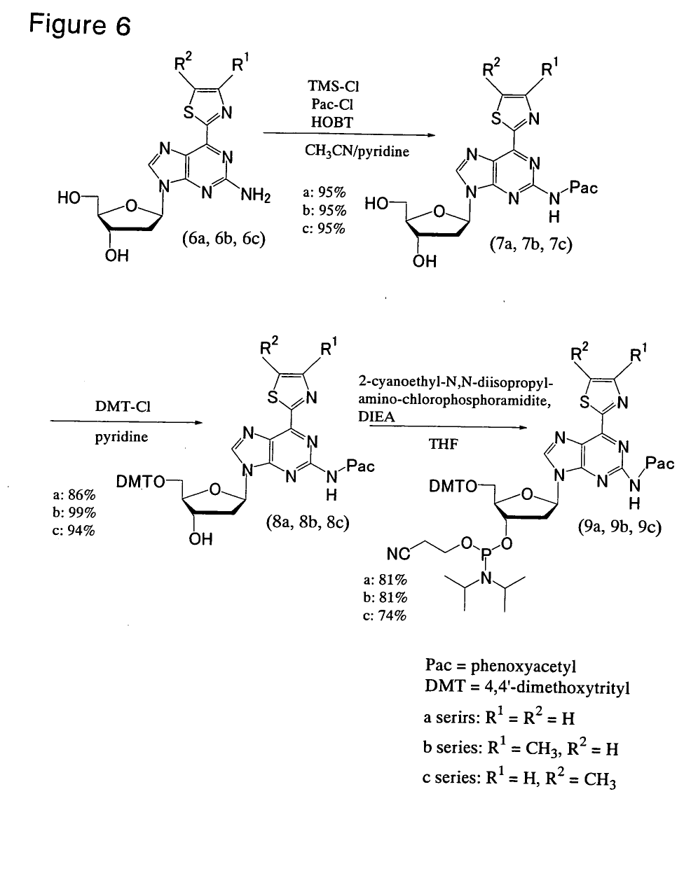

### 3) Synthesis of 2-phenoxyacetylamino-6-(2-thiazolyl)-9-(2-deoxy-β-D-ribofuranosyl)purine (Compound 7a) (FIG. 6)

2-Amino-6-(2-thiazolyl)-9-(2-deoxy-β-D-ribofuranosyl)purine (Compound 6a) synthesized in 2) (150 mg, 0.45 mmol) was dissolved in pyridine (2.2 ml). To this solution, trimethylsilyl chloride (TMS-Cl) (423 μl, 3.3 mmol) was added and stirred at room temperature for 25 minutes (Solution A). Separately, 1-hydroxybenzotriazole (HOBT) (108 mg, 0.8 mmol) in pyridine (221 μl) and acetonitrile (221 μl) was cooled at 0° C. To this solution, phenoxyacetyl chloride (Pac-Cl) (92 μl, 0.67 mmol) was added and stirred at 0° C. for 5 minutes (Solution B).

Solution A cooled to 0° C. was added to Solution B on ice and stirred at room temperature for 12 hours. After the reaction mixture was cooled on ice to 0° C., concentrated aqueous ammonia (220 μl) and H₂O (220 μl) were added and stirred at 0° C. for 10 minutes. The reaction mixture was partitioned by addition of ethyl acetate and water, and the organic layer was dried over Na₂SO₄ and then evaporated under reduced pressure to remove the solvent. The residue was purified by silica gel column chromatography (eluted with 5% MeOH in CH₂Cl₂) to give the desired product 2-phenoxyacetylamino-6-(2-thiazolyl)-9-(2-deoxy-β-D-ribofuranosyl)purine (Compound 7a) (200 mg, 95%).

¹H-NMR (270 MHz, DMSO-d₆) δ 2.34 (m, 1H), 2.78 (m, 1H), 3.57 (m, 2H), 3.88 (m, 1H), 4.45 (m, 1H), 4.93 (t, 1H, J=5.3 Hz), 5.12 (s, 2H), 5.34 (d, 1H, J=4.0 Hz), 6.42 (t, 1H, J=6.6 Hz), 6.95 (m, 3H), 7.30 (t, 2H, J=7.5), 8.12 (d, 1H, J=3.1 Hz), 8.21 (d, 1H, J=3.1), 8.79 (s, 1H), 10.96 (s, 1H);

¹³C-NMR (68 MHz, DMSO-d₆) δ 45.67, 61.46, 67.33, 70.49, 83.28, 87.91, 114.34, 120.71, 125.14, 126.01, 129.30, 145.13, 145.28, 146.85, 151.89, 153.77, 157.75, 162.75, 167.55;

HRMS (FAB, 3-NBA matrix) calculated for C₂₁H₂₁N₆O₅S (M+1): 469.1294, found: 469.1300; TLC Rf=0.25 (CH₂Cl₂:MeOH=9:1, v/v).

### 4) Synthesis of 2-phenoxyacetylamino-6-(2-thiazolyl)-9-(2-deoxy-5-O-dimethoxytrityl-β-D-ribofuranosyl)purine (Compound 8a) (FIG. 6)

2-Phenoxyacetylamino-6-(2-thiazolyl)-9-(2-deoxy-β-D-ribofuranosyl)purine (Compound 7a) synthesized in 3) (94 mg, 0.20 mmol) was azeotropically dried three times with pyridine. 4,4′-Dimethoxytrityl chloride (75 mg, 1.1 molar equivalents) and pyridine (2.0 ml) were then added and stirred at room temperature for 20 hours. After the reaction mixture was partitioned by addition of ethyl acetate and 5% NaHCO₃, the organic layer was washed twice with saturated aqueous sodium chloride. After the organic layer was dried over Na₂SO₄ and concentrated, the residue was purified by silica gel column chromatography (eluted with CH₂Cl₂:EtOAc=1:1, v/v) to give the desired product 2-phenoxyacetylamino-6-(2-thiazolyl)-9-(2-deoxy-5-O-dimethoxytrityl-β-D-ribofuranosyl)purine (133 mg, 86%) (Compound 8a).

¹H-NMR (270 MHz, CDCl₃) δ 2.39 (d, 1H, J=3.8), 2.64 (m, 1H), 2.89 (m, 1H), 3.33-3.48 (m, 2H), 3.72 (s, 6H), 4.17 (m, 1H), 4.43 (bs, 2H), 4.88 (m, 1H), 6.55 (t, 1H, J=6.4 Hz), 6.74 (dd, 4H, J=2.4, 9.0), 7.03 (d, 2H, J=8.7), 7.08-7.37 (m, 12H), 7.64 (d, 1H, J=3.1 Hz), 8.21 (d, 1H, J=3.1), 8.29 (s, 1H);

¹³C-NMR (68 MHz, CDCl₃) δ 41.08, 55.16, 64.06, 67.76, 72.58, 84.27, 86.38, 86.91, 113.01, 114.83, 122.33, 123.60, 126.75, 127.17, 127.73, 127.98, 129.71, 129.85, 129.89, 133.53, 135.61, 144.39, 144.66, 145.71, 148.03, 151.19, 153.71, 156.74, 158.26, 163.42, 166.30;

HRMS (FAB, 3-NBA matrix) calculated for C₄₂H₂₃₉N₆O₇S (M+1): 771.2601, found: 771.2633; TLC Rf=0.22 (CH₂Cl₂:MeOH=20:1, v/v).

### 5) Synthesis of 2-phenoxyacetylamino-6-(2-thiazolyl)-9-[2-deoxy-5-O-dimethoxytrityl-3-O—(N,N-diisopropyl-2-cyanoethylphosphoramidyl)-β-D-ribofuranosyl)purine (Compound 9a) (FIG. 6)

2-Phenoxyacetylamino-6-(2-thiazolyl)-9-(2-deoxy-5-O-dimethoxytrityl-β-D-ribofuranosyl)purine (Compound 8a) synthesized in 4) (130 mg, 0.17 mmol) was azeotropically dried three times with pyridine and then three times with THF, followed by addition of THF (850 μl) and diisopropylethylamine (DIEA) (44 μl, 1.5 molar equivalents). To this mixture, 2-cyanoethyl-N,N-diisopropylaminochlorophosphoramidite (41 μl, 1.1 molar equivalents) was added while stirring at room temperature.

The reaction mixture was stirred at room temperature for 1.5 hours, followed by addition of methanol (50 μl). This mixture was partitioned by addition of an ethyl acetate-triethylamine mixture (EtOAc:TEA=20:1, v/v, 10 ml) and 5% NaHCO₃ (10 ml), and the organic layer was washed three times with saturated aqueous sodium chloride. After the organic layer was dried over Na₂SO₄ and concentrated, the residue was purified by silica gel column chromatography (eluted with CH₂Cl₂:hexane=2:3, v/v, 2% TEA) to give the desired product 2-phenoxyacetylamino-6-(2-thiazolyl)-9-(2-deoxy-5-β-dimethoxytrityl-3-O—(N,N-diisopropyl-2-cyanoethylphosphoramidyl)-β-D-ribofuranosyl)purine (Compound 9a) (133 mg, 81%, white foam).

¹H-NMR (270 MHz, CDCl₃) δ 1.09-1.18 (m, 12H), 2.44 (t, 1H, J=6.6), 2.62 (t, 1H, J=6.6), 2.75 (m, 1H), 2.89 (m, 1H), 3.35-3.85 (m, 12H), 4.30 (m, 1H), 4.82 (m, 3H), 6.52 (t, 1H, J=6.4), 6.74 (m, 4H), 7.03-7.37 (m, 14H), 7.64 (d, d, 1H, J=3.1), 8.21 (d, 1H, J=3.1), 8.33, 8.34 (s, s, 1H);

³¹P-NMR (109 MHz, CDCl₃) δ 149.57;

HRMS (FAB, 3-NBA matrix) calculated for C₅₁H₅₆N₈O₈SP (M+1): 971.3679, found: 971.3696; TLC Rf=0.20 and 0.26 (diastereoisomer) (CH₂Cl₂:hexane=3:2, v/v, 2% TEA).

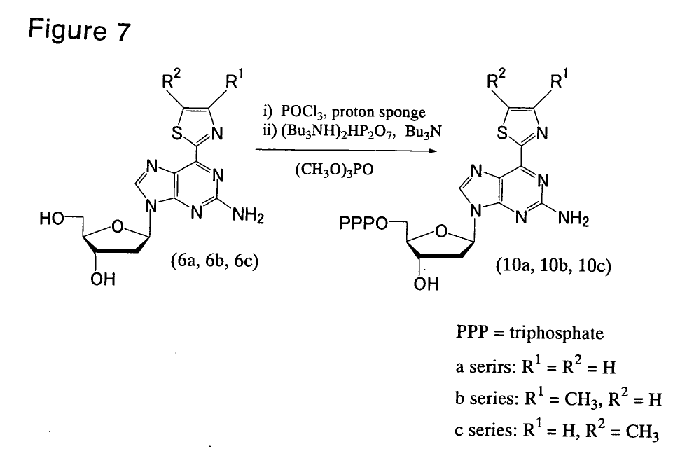

### 6) Synthesis of 2-amino-6-(2-thiazolyl)-9-(2-deoxy-β-D-ribofuranosyl)purine 5′-triphosphate (Compound 10a) (FIG. 7)

2-Amino-6-(2-thiazolyl)-9-(2-deoxy-β-D-ribofuranosyl)purine (Compound 6a) synthesized in 2) (33 mg, 0.10 mmol) was azeotropically dried three times with toluene, followed by addition of proton sponge (32 mg, 0.15 mmol) and trimethyl phosphate (500 μl). While stirring this mixture on ice, POCl₃ (12 μl, 0.13 mmol) was added dropwise thereto.

After stirring at 0° C. for 2 hours, tri-n-butylamine (Bu₃N) (120 μl, 0.5 mmol) and bis-tributylammonium pyrophosphate ((Bu₃NH)₂HP₂O₇) (0.5 M in DMF solution, 1.0 ml, 0.5 mmol) were added and stirred at 0° C. for 10 minutes. To this mixture, triethylammonium bicarbonate (0.5 M solution, 500 μl) and then 5 ml H₂O were added, and the reaction mixture was purified by DEAE Sephadex A-25 column chromatography (1.5×30 cm, 50 mM-1.5 M TEAB, linear gradient) (crude product, 32 mg). The resulting product was finally purified by RP-HPLC (4.6×250 mm, MICRA Scientific Inc. Synchropak RPP, flow rate: 1 ml/min, 0%-30% CH₃CN in 100 mM TEAA, 10 minutes, linear gradient) to give the desired product 2-amino-6-(2-thiazolyl)-9-(2-deoxy-β-D-ribofuranosyl)purine 5′-triphosphate (Compound 10a).

¹H-NMR (270 MHz, D₂O) δ 1.11 (t, 27H, J=7.3 Hz), 2.42 (m, 1H), 2.77 (m, 1H), 3.03 (q, 18H, J=7.3 Hz), 4.09 (m, 3H), 4.28 (m, 1H), 6.34 (t, 1H, J=6.5 Hz), 7.76 (d, 1H, J=2.5 Hz), 7.99 (d, 1H, J=2.5), 8.36 (s, 1H);

³¹P-NMR (109 MHz, D₂O) δ −22.52 (t, 1H, J=19.8 Hz), −10.65 (d, 1H, J=20.7 Hz), −9.69 (d, 1H, J=18.3 Hz);

ESI-MS calculated for C₁₃H₁₆N₆O₁₂P₃S (M−1): 572.98, found: 572.94.

### Example 2

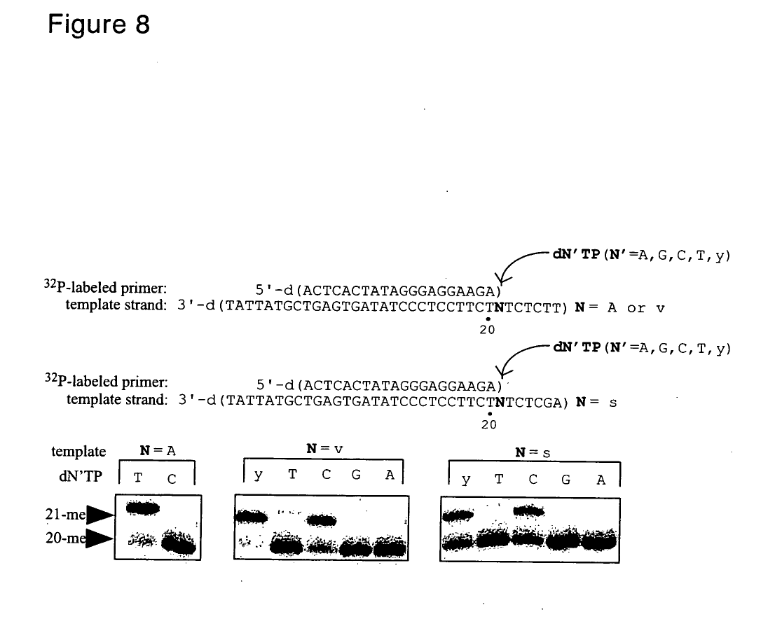

### Site-Selective Introduction of Unnatural Bases During Replication—Klenow Fragment-mediated Single Nucleotide Insertion Experiment (FIG. 8)

In this example, E. coli-derived DNA polymerase I lacking 3′→5′ exonuclease activity, i.e., Klenow fragment (KF exo⁻) was used to make a comparison of the efficiency for single nucleotide incorporation during replication (i.e., incorporation of 2-oxo-(1H)pyridine (y) into DNA) between v-y base pair (the present invention) and s-y base pair (control).

More specifically, Large fragment of DNA polymerase Exonuclease-free Klenow enzyme (Cloned) (Amersham USB) and 10× reaction buffer attached thereto (500 mM Tris-HCl pH 7.5, 100 mM MgCl₂, 10 mM DTT, 0.5 mg/ml BSA) were used. The enzyme concentration of KF exo⁻ was determined using a Bio-Rad Protein Assay kit (BioRad) for each lot purchased.

The primer used in the reaction was a synthetic oligonucleotide having the following sequence.

5′-actcactatagggaggaaga-3′ (SEQ ID NO: 1, FIG. 8)

The primer for use in the reaction was pre-labeled at its 5′-end using T4 polynucleotide kinase (TaKaRa) and [α-³²P]ATP, and then purified by gel electrophoresis.

The template DNA used was a synthetic oligonucleotide having the following sequence.

5′-ttctctntcttcctccctatagtgagtcgtattat-3′ (n = a
or v) (SEQ ID NO: 2, FIG. 8)
or
5′-agctctntcttcctccctatagtgagtcgtattat-3′ (n = s)
(SEQ ID NO: 3, FIG. 8)

Reaction conditions: a mixed solution of template DNA (20 μM, 1 μl), the primer whose 5′-end was labeled with ³²P (5 μM, 4 μl) and 10× reaction buffer (1 μl) was heated at 95° C. for 3 minutes and then annealed by quenching to form a duplex between the template DNA and the primer. A Klenow fragment solution (1 μM) diluted with enzyme dilution buffer (50 mM phosphate buffer pH 7, 50% glycerol, 1 mM DTT) was added in a volume of 2 μl and incubated at 37° C. for 2 minutes, followed by addition of 2 μl dNTP solution (any one of A, G, C, T or y shown in FIG. 8) (100 μM) to start the reaction. After incubation at 37° C. for 2 minutes, 10 μl of a 10 M urea-containing TBE solution was added and heated at 75° C. for 3 minutes to stop the reaction. The reaction conditions are summarized as follows: template/primer 2 μM; KF exo⁻ 200 nM; dNTP 20 μM; reaction at 37° C. for 2 minutes.

Aliquot parts of the reaction solutions were electrophoresed on a 20% polyacrylamide-7 M urea gel and the reaction products were analyzed with a bioimaging analyzer (BAS2500, Fuji Photo Film Co., Ltd., Japan). The results obtained are shown in FIG. 8. In the single-base incorporation experiment using a nucleic acid containing the nucleotide v of the present invention as a template, y was selectively incorporated at a site corresponding to v in the complementary DNA strand. However, C was also incorporated although in a small amount. The incorporation selectivity of v (the present invention) opposite y was comparable to the selectivity of s.

### Example 3

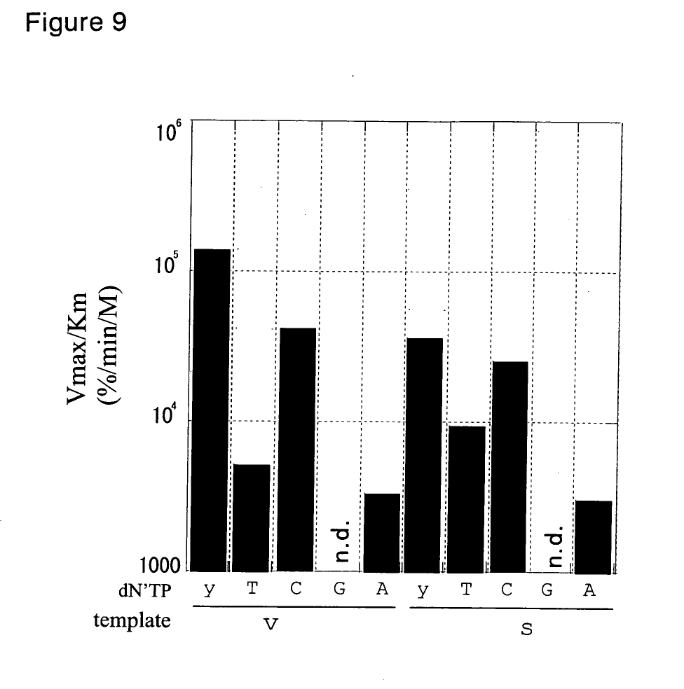

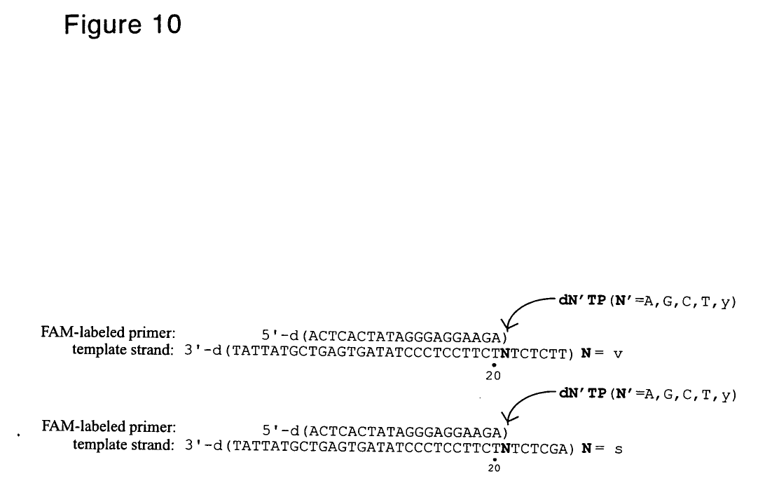

### Site-selective Introduction of Unnatural Bases During Replication—Analysis of Reaction Rate Constants for Klenow fragment-mediated Single Nucleotide Insertion Reaction (FIGS. 9-10)

This example was intended to analyze reaction rate constants in the same Klenow fragment-mediated single nucleotide insertion reaction as shown in Example 2.

More specifically, the reaction primer used was a primer whose 5′-end was fluorescently labeled with 6-FAM (SEQ ID NO: 1, FIG. 10). The primer whose 5′-end was fluorescently labeled was purchased from Applied Biosystems among those commercially available as custom fluorescent primers for GeneScan, and purified by gel electrophoresis. The analysis of reaction products was performed with a DNA sequencer (Applied Biosystems; model ABI377).

Reaction conditions: Template DNA (SEQ ID NO: 2 or 3) (10 μM) and the fluorescently-labeled primer (10 μM), each of which had been dissolved in 2× reaction buffer (100 mM Tris-HCl pH 7.5, 20 mM MgCl₂, 2 mM DTT, 100 μg/ml BSA), were heated at 95° C. for 3 minutes and then annealed by quenching to form a duplex between the template and the primer. After this duplex DNA solution was dispensed in 5 μl aliquots, a KF exo⁻ solution (15-250 nM) diluted with enzyme dilution buffer was added in a volume of 2 μl, followed by incubation at 37° C. for 2 minutes to form a DNA-enzyme complex. The resulting solution was supplemented with 3 μl dNTP solution (any one of A, G, C, T or y) (100 μM-7 mM) and enzymatically reacted at 37° C. (1.5-20 minutes). The reaction was stopped by adding 10 μl of a 95% formamide solution containing 20 mM EDTA (stop solution) and heating at 75° C. for 3 minutes.

The reaction conditions are summarized as follows: 5 μM template-primer duplex, 3-50 nM enzyme and 30-2100 μM dNTP are used in a solution (10 μl). The solution (10 μl) contains 50 mM Tris-HCl (pH 7.5), 10 mM MgCl₂, 1 mM DTT and 0.05 mg/ml BSA. The reaction is performed at 37° C. for 1.5-20 minutes.

After aliquot parts of the reaction solutions were diluted 50-fold with the stop solution, the thus diluted reaction solutions (0.5 μl) were each mixed with 3 μl loading solution (deionized formamide:50 mg/mL blue dextran solution containing 25 mM EDTA=5:1), heated at 90° C. for 2 minutes and then quenched on ice. Aliquots (about 0.5 μl) of the resulting solutions were loaded on every other lane of a sequence gel and electrophoresed. The sequence gel (36 cm WTR) has the following composition: 6 M urea, 8% polyacrylamide (acrylamide:bisacrylamide=19:1), and 0.5×TBE. The electrophoresis buffer used was 0.5×TBE. The run module used was GS Run 36C-2400. The electrophoresis time was set to about 1 hour, and peak patterns of the reaction products were analyzed and quantified using GeneScan Software (Version 3.0).

Using the peak heights of the unreacted primer fragment and single nucleotide-inserted DNA fragments, the percentage of each primer elongated by a single nucleotide was determined to calculate enzymological parameters Kₘ and Vₘₐₓ from Hanes-Woolf plots (Goodman, M. F., Creighton, S., Bloom, L. B., Petruska, J. Crit. Rev. Biochem. Mol. Biol., 28, 83-126 (1993)). The results obtained are shown in Table 1 and FIG. 9.

TABLE 1

| | nucleoside | | | |
|---|---|---|---|---|
| Template | triphosphate | Km | Vmax | efficiency |
| (N) | (N′) | (μM) | (% min⁻¹)^{C} | (Vmax/Km)^{d} |
| v | y | 290(180)^{a} | 40(21) | 1.4 × 10⁵ |
| v | T | 390(200) | 2.0(0.7) | 5.1 × 10³ |
| v | C | 540(60) | 22(5) | 4.1 × 10⁴ |
| v | G | n.d.^{b} | n.d.^{b} | |
| v | A | 110(10) | 0.36(0.08) | 3.3 × 10³ |
| s | y | 260(70) | 9.4(2.5) | 3.6 × 10⁴ |
| s | T | 320(30) | 3.0(0.3) | 9.4 × 10³ |
| s | C | 590(230) | 15(5) | 2.5 × 10⁴ |
| s | G | n.d.^{b} | n.d.^{b} | |
| s | A | 86(12) | 0.26(0.06) | 3.0 × 10³ |

^{a}= Standard deviation is shown in parentheses.
^{b}= Not detected. The reaction was very inefficient and resulted in no accurate measured value.
^{c}= This value was normalized to enzyme concentration (20 nM) relative to various enzyme concentrations used.
^{d}= The unit of this term is % min⁻¹M⁻¹.

As shown in Table 1 and FIG. 9, the incorporation efficiency of y opposite v was Vmax/Km=1.4×10⁵, which was comparable to the incorporation efficiency between natural bases A/T. It was also about 4-fold higher than the incorporation efficiency of y opposite s (Vmax/Km=3.6×10⁴). In terms of selectivity, the incorporation efficiency of y opposite v was about 3-fold higher than that of c incorporation (Vmax/Km=4.1×10⁴) and 20-fold or more higher than that of t incorporation (Vmax/Km=5.1×10³).

### Example 4

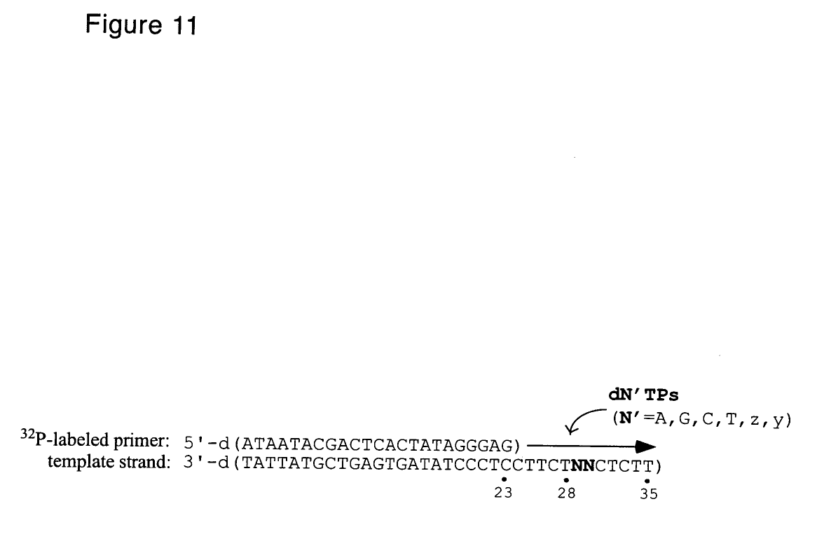

### Site-selective Introduction of Unnatural Bases During Replication—Klenow Fragment-mediated Elongation Reaction (FIGS. 11-13)

This example was intended to study selective introduction of y at a site corresponding to v in the complementary DNA strand during Klenow fragment-mediated elongation reaction, rather than single-base incorporation. The reaction primer DNA and template DNA used are shown below.

Primer
5′-ataatacgactcactatagggag-3′ (SEQ ID NO: 4,
FIG. 11)
Template DNA
5′-ttctcnntcttcctccctatagtgagtcgtattat-3′ (nn =
ta, tv, ts, vv or ss) (SEQ ID NO: 5, FIG. 11)

As in the case of Example 2, the primer was pre-labeled at its 5′-end using [α-³²P]ATP and then purified by gel electrophoresis. In Experiments 2 and 3, the base y is incorporated as the first base of elongation from the primer. In contrast, in this experiment, v in the template is located at a position corresponding to the severalth base elongated from the primer, and it is therefore possible to study the introduction of y at a site corresponding to v in the complementary DNA strand during elongation reaction.

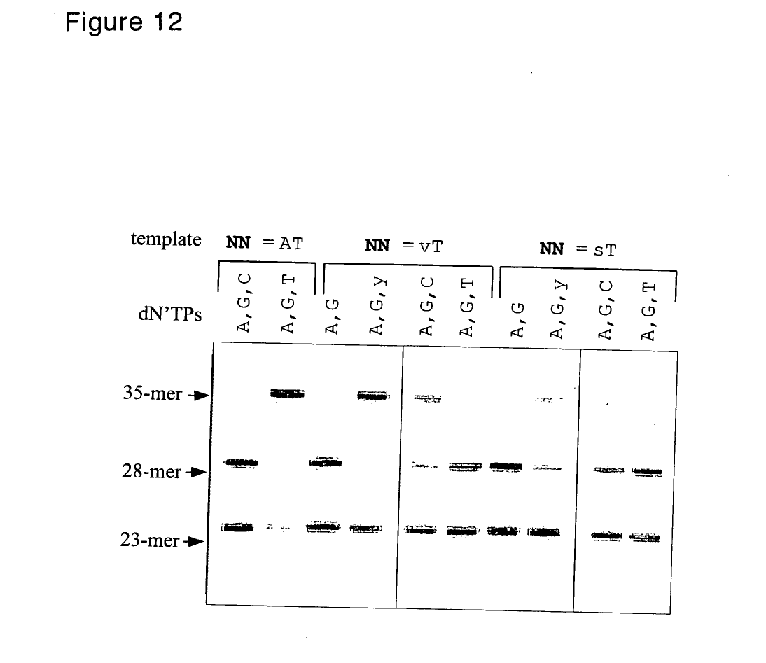

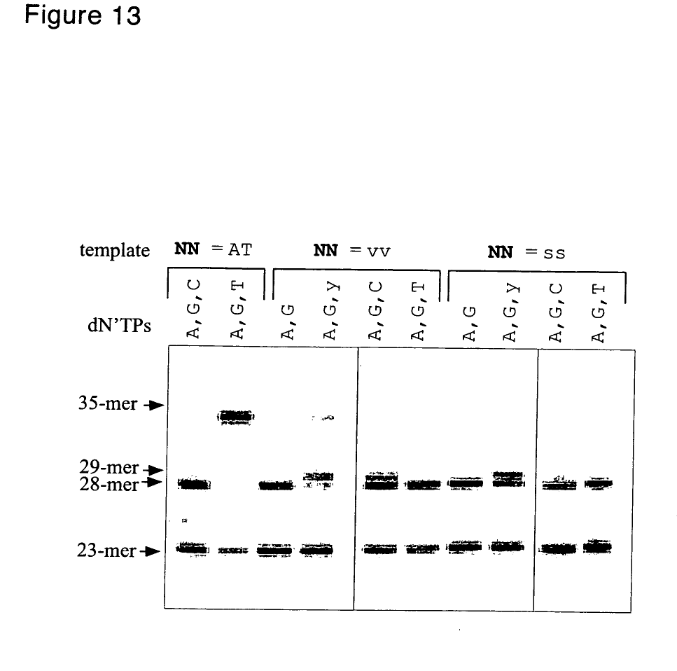

The template DNA (400 nM) and the primer whose 5′-end was labeled with ³²P (FIG. 11) (400 nM), each of which had been dissolved in 2× reaction buffer (20 mM Tris-HCl pH 7.5, 14 mM MgCl₂, 0.2 mM DTT), were heated at 95° C. for 3 minutes and then annealed by quenching to form a duplex. After this duplex DNA solution was dispensed in 5 μl aliquots, 2 μl of a dNTP solution (a combination shown in each lane of FIG. 12 or 13) (50 μM) and 3 μl (0.15 units) of Klenow fragment (KF exo⁺; Cloned Klenow Fragment for sequencing (Large Fragment E. coli DNA Polymerase I), purchased from TaKaRa) diluted with water were added to start the enzymatic reaction at 37° C. After incubation for 3 minutes, the enzymatic reaction was stopped by adding 10 μl of a 10 M urea-containing TBE solution and heating at 75° C. for 3 minutes. The reaction conditions are summarized as follows: template/primer 200 nM; KF exo⁻ 0.015U/μl; dNTPs 10 μM; reaction at 37° C. for 3 minutes.

Aliquot parts of the reaction solutions were electrophoresed on a 15% polyacrylamide-7 M urea gel and the reaction products were analyzed with a bioimaging analyzer (BAS2500, Fuji Photo Film Co., Ltd., Japan). The results obtained are shown in FIGS. 12 and 13. In FIG. 12, when a comparison is made between the lane of substrates A, G and y at NN=vT and the lane of substrates A, G and y at NN=sT, the 35-mer band intensity indicates that the v-y base pair also has higher replication efficiency than the s-y base pair in elongation reaction during replication. Further, the replication efficiency was also studied in the case of containing two adjacent v (the present invention) or s (control) (FIG. 13). As shown in FIG. 13, when using template DNA containing two adjacent s, there was a significant reduction in the incorporation efficiency of two y substrates at corresponding positions, and little 35-mer product was obtained (lane indicated as A, G, y at NN=ss). In contrast, the figure indicates that v allows replication to proceed even if two v are adjacent to each other, thereby giving a product in which two y substrates are incorporated into the complementary DNA strand (lane indicated as A, G, y at NN=vv).

### Example 5

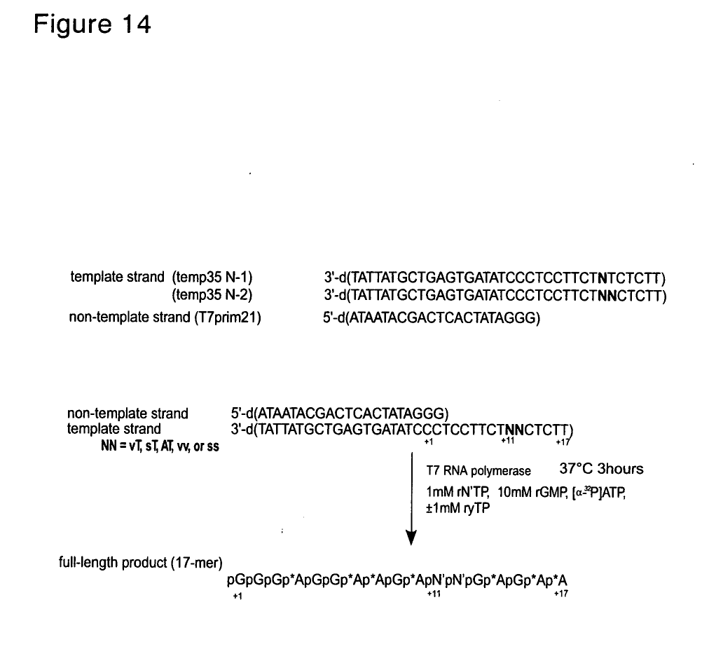

### Site-selective Introduction of ryTP into RNA through Transcription (FIGS. 14-16)

This example was intended to study site-selective introduction of ryTP into RNA through transcription reaction. More specifically, 35-mer DNAs containing v and s (temp35N-1 and temp35N-2 shown in SEQ ID NOs: 2 and 5, respectively) were each used as a template in transcription reaction with T7 RNA polymerase. The DNA primer required for the transcription reaction had the following sequence.

T7prim21; 21-mer
5′-ataatacgactcactataggg-3′ (SEQ ID NO: 6,
FIG. 14)

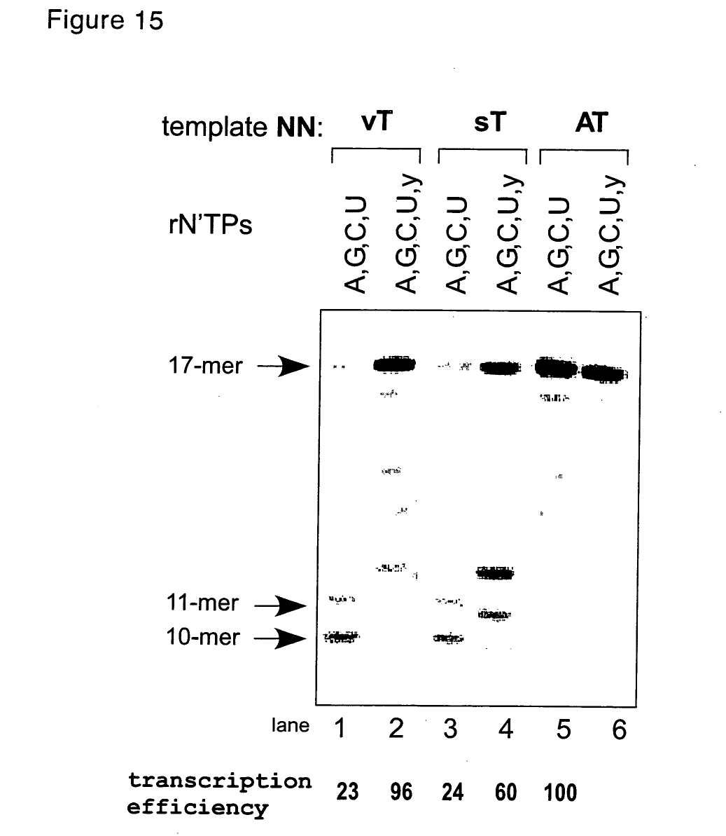

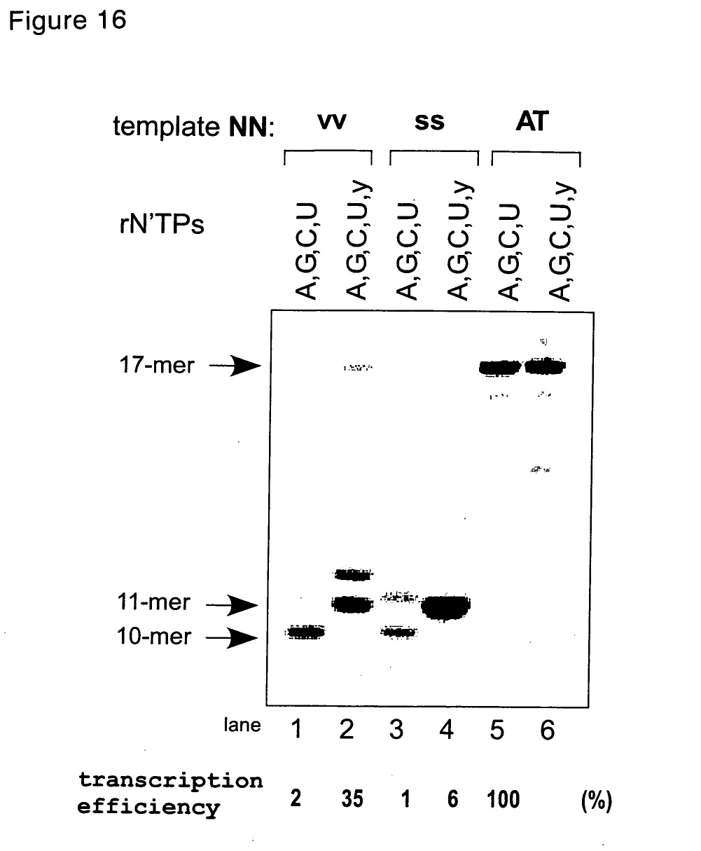

A template and T7prim21 were mixed in 10 mM Tris-HCl (pH 7.6) containing 10 mM NaCl and annealed into a double-stranded form for use in the transcription reaction (FIG. 14). The T7 transcription reaction was performed on 20 μl scale using an enzyme from TAKARA SHUZO CO., LTD [T. Ohtsuki et al., Proc. Natl. Acad. Sci. USA, 98, 4922-4925 (2001)]. More specifically, the transcription reaction was accomplished by incubation at 37° C. for 3 hours in a reaction solution containing 40 mM Tris-HCl pH 8.0, 5 mM DTT, 24 mM MgCl₂, 2 mM spermidine, 0.01% TritonX-100, 10 mM GMP, 1 mM NTPs (N=G, C, U, with or without ryTP, as shown in FIGS. 15 and 16), 2 μCi [α-³²P]ATP, 2 μM double-stranded DNA (template and T7prim21), and 2.5 U/μl T7 RNA polymerase (TaKaRa). If the transcription reaction proceeds completely, an RNA product having the following full-length sequence can be obtained.

5′-gggaggaaganngagaa-3′ (nn = ua, ya or yy) (SEQ
ID NO: 7, FIG. 14)

The reaction solutions were each supplemented with an equal volume of a 10 M urea-containing BPBdye solution and heated at 75° C. for 3 minutes to stop the reaction, followed by electrophoresis on a 20% polyacrylamide-7 M urea gel to confirm products of the transcription reaction. The [α-³²P]ATP-labeled reaction products were analyzed with a bioimaging analyzer (BAS2500, Fuji Photo Film Co., Ltd., Japan). The results obtained are shown in FIGS. 15 and 16. The incorporation efficiency of the substrate y into RNA when using the sT-containing template in transcription with T7 RNA polymerase (FIG. 14) was about 50% to 60%, as compared to natural base pairing (AT). In contrast, when using the vT-containing template, the incorporation efficiency of the substrate y was 96%, which was as high as that of natural base pairing (FIG. 15).

Moreover, in a case where two s were adjacent to each other in a template (NN=ss), no RNA incorporating two y was obtained. In contrast, when two v were adjacent to each other in a template (NN=vv), transcription proceeded and two y substrates were incorporated into RNA although the transcription efficiency was about 30% (FIG. 16).

### Example 6

### Synthesis of a 2-amino-6(4-methyl-2-thiazolyl)-9-(2-deoxy-β-D-ribofuranosyl)purine derivative (FIGS. 5-7)

### 1) Synthesis of 2-Tributyltin-4-Methylthiazole (Compound 3B) (FIG. 5)

Under an argon atmosphere, 4-methylthiazole (Compound 2b) (455 μl, 5.0 mmol) was added to diethyl ether (25 ml) which had been cooled to −78° C. Subsequently, n-butyllithium (1.58 M in hexane, 3.2 ml, 5.0 mmol) was added dropwise and stirred at −78° C. for 30 minutes. To this solution, tributyltin chloride (1.5 ml, 5.5 mmol) was added dropwise at −78° C., and the mixture was normally warmed while stirring up to room temperature (30 minutes). After this reaction mixture was washed three times with saturated aqueous sodium chloride, the organic layer was dried over MgSO₄ and evaporated to remove the solvent, thereby obtaining 2-tributyltin-4-methylthiazole (Compound 3b) (yellow liquid). Compound 3b was used as such in the subsequent reaction without further purification.

### 2) Synthesis of 2-amino-6-(4-methyl-2-thiazolyl)-9-(2-deoxy-β-D-ribofuranosyl)purine (Compound 6b) (FIG. 5)

Starting with 2-tributyltin-4-methylthiazole (Compound 3b) synthesized in 1) and 2-amino-6-tosyloxy-9-(2-deoxy-3,5-di-O-tert-butyldimethylsilyl-β-D-ribofuranosyl)purine (Compound 4), the same procedure as used for synthesis of Compound 6a in Example 1 was repeated to give the desired product 6b in 78% yield for 2 steps.

¹H-NMR (270 MHz, DMSO-d₆) δ 2.27 (m, 1H), 2.49 (s, 3H), 2.65 (m, 1H), 3.56 (m, 2H), 3.85 (m, 1H), 4.39 (m, 1H), 4.96 (br s, 1H), 5.30 (br s, 1H), 6.30 (t, 1H, J=6.8 Hz), 6.73 (br s, 2H), 7.57 (s, 1H), 8.40 (s, 1H);

¹³C-NMR (68 MHz, DMSO-d₆) δ 17.08, 61.55, 70.58, 82.56, 87.57, 118.76, 122.64, 141.51, 147.31, 154.02, 154.69, 159.70, 162.79;

HRMS (FAB, 3-NBA matrix) calculated for C₁₄H₁₇N₆O₃S (M+1): 349.1083, found: 349.1063;

UV-vis (in EtOH) λmax=232 nm (ε=17600), 311 nm (ε=8260), 361 nm (ε=9020), λmin=267 nm (λ=2750), 334 nm (λ=6740);

TLC Rf=0.20 (CH₂Cl₂:MeOH=9:1, v/v).

### 3) Synthesis of 2-phenoxyacetylamino-6-(4-methyl-2-thiazolyl)-9-(2-deoxy-β-D-ribofuranosyl)purine (Compound 7b) (FIG. 6)

Starting with 2-amino-6-(4-methyl-2-thiazolyl)-9-(2-deoxy-β-D-ribofuranosyl)purine (Compound 6b) synthesized in 2), the same procedure as used for synthesis of Compound 7a in Example 1 was repeated to give the desired product 7b in 95% yield.

¹H-NMR (300 MHz, DMSO-d₆) δ 2.35 (m, 1H), 2.51 (s, 3H), 2.80 (m, 1H), 3.59 (m, 2H), 3.89 (m, 1H), 4.47 (m, 1H), 4.92 (t, 1H, J=5.4 Hz), 5.12 (s, 2H), 5.35 (d, 1H, J=4.1 Hz), 6.43 (t, 1H, J=6.7 Hz), 6.97 (m, 3H), 7.31 (t, 2H, J=7.5 Hz), 7.70 (s, 1H), 8.78 (s, 1H), 10.96 (s, 1H);

¹³C-NMR (75 MHz, DMSO-d₆) δ 17.01, 61.49, 67.33, 70.54, 83.38, 87.99, 114.46, 120.13, 120.85, 126.13, 129.44, 145.31, 147.03, 152.09, 153.84, 154.75, 157.95, 161.77, 167.65;

HRMS (FAB, 3-NBA matrix) calculated for C₂₂H₂₃N₆O₅S (M+1): 483.1451, found: 483.1414;

TLC Rf=0.23 (CH₂Cl₂:MeOH=9:1, v/v).

### 4) Synthesis of 2-phenoxyacetylamino-6-(4-methyl-2-thiazolyl)-9-(2-deoxy-5-O-dimethoxytrityl-β-D-ribofuranosyl)purine (Compound 8b) (FIG. 6)

Starting with 2-phenoxyacetylamino-6-(4-methyl-2-thiazolyl)-9-(2-deoxy-β-D-ribofuranosyl)purine (Compound 7b) synthesized in 3), the same procedure as used for synthesis of Compound 8a in Example 1 was repeated to give the desired product 8b in 99% yield.

¹H-NMR (270 MHz, CDCl₃) δ 2.65 (s, m, 3H, 1H), 2.86 (m, 1H), 3.34-3.47 (m, 2H), 3.72 (s, 6H), 4.18 (m, 1H), 4.69 (br s, 2H), 4.87 (m, 1H), 6.59 (t, 1H, J=6.3 Hz), 6.74 (dd, 4H, J=2.0, 8.9 Hz), 7.00-7.38 (m, 15H), 8.28 (s, 1H), 9.12 (br s, 1H);

¹³C-NMR (68 MHz, CDCl₃) δ 17.73, 40.49, 55.21, 60.41, 63.98, 67.90, 72.47, 84.43, 86.35, 86.49, 113.07, 114.86, 119.27, 122.26, 123.63, 126.79, 127.31, 127.74, 127.99, 129.72, 129.89, 135.57, 135.61, 144.41, 148.27, 149.72, 151.39, 153.40, 155.90, 156.92, 158.35, 162.02, 166.11;

HRMS (FAB, 3-NBA matrix) calculated for C₄₃H₄₁N₆O₇S (M+1): 785.2757, found: 785.2715;

TLC Rf=0.48 (CH₂Cl₂:MeOH=9:1, v/v).

### 5) Synthesis of 2-phenoxyacetylamino-6-(4-methyl-2-thiazolyl)-9-[2-deoxy-5-O-dimethoxytrityl-3-O—(N,N-diisopropyl-2-cyanoethylphosphoramidyl)-β-D-ribofuranosyl]purine (Compound 9b) (FIG. 6)

Starting with 2-phenoxyacetylamino-6-(4-methyl-2-thiazolyl)-9-(2-deoxy-5-O-dimethoxytrityl-β-D-ribofuranosyl)purine (Compound 8b) synthesized in 4), the same procedure as used for synthesis of Compound 9a in Example 1 was repeated to give the desired product 9b in 81% yield.

¹H-NMR (300 MHz, CDCl₃) δ 1.09-1.13 (m, 12H), 2.38 (t, 1H, J=6.5 Hz), 2.57 (t, 1H, J=6.5 Hz), 2.60 (s, 3H), 2.70 (m, 1H), 2.81 (m, 1H), 3.34 (dd, 2H, J=4.1, 13.6 Hz), 3.68 (s, 6H), 3.49-3.83 (m, 4H), 4.24 (m, 1H), 4.69 (m, 1H), 4.74 (br s, 2H), 6.46 (t, 1H, J=6.4 Hz), 6.69 (m, 4H), 6.96-7.33 (m, 15H), 8.25, 8.27 (s, s, 1H), 8.94 (br s, 1H);

³¹P-NMR (121 MHz, CDCl₃) δ 149.09;

HRMS (FAB, 3-NBA matrix) calculated for C₅₂H₅₈N₈O₈PS (M+1): 985.3836, found: 985.3973;

TLC Rf=0.38 and 0.25 (diastereoisomer) (CH₂Cl₂:hexane=3:2, v/v, 2% TEA).

### Example 7

### Synthesis of a 2-amino-6(5-methyl-2-thiazolyl)-9-(2-deoxy-β-D-ribofuranosyl)purine derivative (FIGS. 5-7)

### 1) Synthesis of 2-Tributyltin-5-Methylthiazole (Compound 3C) (FIG. 5)

Starting with 5-methylthiazole (Compound 2c), the same procedure as used for synthesis of Compound 3b in Example 6 was repeated to give the desired product 3c, which was used as such in the subsequent reaction without further purification.

### 2) Synthesis of 2-amino-6-(5-methyl-2-thiazolyl)-9-(2-deoxy-β-D-ribofuranosyl)purine (Compound 6c) (FIG. 5)

Starting with 2-tributyltin-5-methylthiazole (Compound 3c) synthesized in 1) and 2-amino-6-tosyloxy-9-(2-deoxy-3,5-di-O-tert-butyldimethylsilyl-β-D-ribofuranosyl)purine (Compound 4), the same procedure as used for synthesis of Compound 6a in Example 1 was repeated to give the desired product 6c in 81% yield for 2 steps.

¹H-NMR (270 MHz, DMSO-d₆) δ 2.26 (m, 1H), 2.54 (s, 3H), 2.64 (m, 1H), 3.55 (m, 2H), 3.84 (m, 1H), 4.38 (m, 1H), 4.96 (t, 1H, J=5.5 Hz), 5.30 (d, 1H, J=4.0 Hz), 6.29 (t, 1H, J=6.8 Hz), 6.68 (br s, 2H), 7.80 (s, 1H), 8.38 (s, 1H);

¹³C-NMR (68 MHz, DMSO-d₆) δ 11.78, 61.57, 70.61, 82.52, 87.57, 122.52, 138.00, 141.44, 142.88, 147.57, 154.61, 159.65, 162.03;

HRMS (FAB, 3-NBA matrix) calculated for C₁₄H₁₇N₆O₃S (M+1): 349.1083, found: 349.1125;

UV-vis (in EtOH) λmax=232 nm (ε=17040), 307 nm (ε=11100), 361 nm (ε=10430), λmin=267 nm (ε=3980), 333 nm (ε=7420);

TLC Rf=0.15 (CH₂Cl₂:MeOH=9:1, v/v).

### 3) Synthesis of 2-phenoxyacetylamino-6-(5-methyl-2-thiazolyl)-9-(2-deoxy-β-D-ribofuranosyl)purine (Compound 7c) (FIG. 6)

Starting with 2-amino-6-(5-methyl-2-thiazolyl)-9-(2-deoxy-β-D-ribofuranosyl)purine (Compound 6c) synthesized in 2), the same procedure as used for synthesis of Compound 7a in Example 1 was repeated to give the desired product 7c in 95% yield.

¹H-NMR (300 MHz, DMSO-d₆) δ 2.35 (m, 1H), 2.59 (s, 3H), 2.79 (m, 1H), 3.59 (m, 2H), 3.89 (m, 1H), 4.45 (m, 1H), 4.93 (t, 1H, J=5.4 Hz), 5.14 (s, 2H), 5.35 (d, 1H, J=3.9 Hz), 6.43 (t, 1H, J=6.7 Hz), 6.97 (m, 3H), 7.31 (t, 2H, J=7.8 Hz), 7.92 (s, 1H), 8.77 (s, 1H), 10.91 (s, 1H);

¹³C-NMR (75 MHz, DMSO-d₆) δ 11.76, 61.49, 67.40, 70.55, 83.32, 87.98, 114.48, 120.83, 125.93, 129.43, 139.52, 143.61, 145.20, 147.17, 152.05, 153.81, 157.95, 160.95, 167.80;

HRMS (FAB, 3-NBA matrix) calculated for C₂₂H₂₃N₆O₅S (M+1): 483.1451, found: 483.1489;

TLC Rf=0.18 (CH₂Cl₂:MeOH=9:1, v/v).

### 4) Synthesis of 2-phenoxyacetylamino-6-(5-methyl-2-thiazolyl)-9-(2-deoxy-5-O-dimethoxytrityl-(3-D-ribofuranosyl)purine (Compound 8c) (FIG. 6)

Starting with 2-phenoxyacetylamino-6-(5-methyl-2-thiazolyl)-9-(2-deoxy-β-D-ribofuranosyl)purine (Compound 7c) synthesized in 3), the same procedure as used for synthesis of Compound 8a in Example 1 was repeated to give the desired product 8c in 94% yield.

¹H-NMR (300 MHz, CDCl₃) δ 2.54 (s, 3H), 2.58 (m, 1H), 2.83 (m, 1H), 3.28-3.42 (m, 2H), 3.67 (s, 6H), 4.12 (m, 1H), 4.68 (bs, 2H), 4.82 (m, 1H), 6.52 (t, 1H, J=6.3 Hz), 6.69 (dd, 4H, J=2.5, 8.8 Hz), 6.96-7.32 (m, 14H), 7.81 (s, 1H), 8.22 (s, 1H), 8.96 (br s, 1H);

¹³C-NMR (75 MHz, CDCl₃) δ 12.28, 40.43, 55.18, 60.39, 64.00, 67.98, 72.56, 84.40, 86.39, 86.54, 113.15, 114.98, 122.36, 123.72, 126.90, 127.10, 127.85, 128.10, 129.83, 129.98, 130.00, 135.69, 135.73, 135.94, 139.39, 144.17, 144.45, 144.53, 148.59, 149.86, 151.44, 153.54, 157.15, 158.51, 161.71;

HRMS (FAB, 3-NBA matrix) calculated for C₄₃H₄₁N₆O₇S (M+1): 785.2757, found: 785.2794;

TLC Rf=0.35 (CH₂Cl₂:MeOH=9:1, v/v).

### 5) Synthesis of 2-phenoxyacetylamino-6-(5-methyl-2-thiazolyl)-9-[2-deoxy-5-O-dimethoxytrityl-3-O—(N,N-diisopropyl-2-cyanoethylphosphoramidyl)-β-D-ribofuranosyl]purine (Compound 9c) (FIG. 6)

Starting with 2-phenoxyacetylamino-6-(5-methyl-2-thiazolyl)-9-(2-deoxy-5-O-dimethoxytrityl-β-D-ribofuranosyl)purine (Compound 8c) synthesized in 4), the same procedure as used for synthesis of Compound 9a in Example 1 was repeated to give the desired product 9c in 74% yield.

¹H-NMR (300 MHz, CDCl₃), 1.10-1.12 (m, 12H), 2.38 (t, 1H, J=6.5 Hz), 2.54 (s, 3H), 2.56 (t, 1H, J=6.5 Hz), 2.68 (m, 1H), 2.83 (m, 1H), 3.32 (m, 2H), 3.67 (s, 6H), 3.47-3.83 (m, 4H), 4.24 (m, 1H), 4.70 (m, 1H), 4.76 (br s, 2H), 6.45 (t, 1H, J=6.6 Hz), 6.69 (m, 4H), 6.96-7.33 (m, 14H), 7.80 (s, 1H), 8.24, 8.25 (s, s, 1H), 8.83 (br s, 1H);

³¹P-NMR (121 MHz, CDCl₃) δ 149.03;

HRMS (FAB, 3-NBA matrix) calculated for C₅₂H₅₈N₈O₈PS (M+1): 985.3836, found: 985.3972;

TLC Rf=0.19 and 0.12 (diastereoisomer) (CH₂Cl₂:hexane=3:2, v/v, 2% TEA).

1. A nucleoside or a nucleotide, or a derivative thereof, which has a 2-amino-6-(2-thiazolyl)purin-9-yl group or a 2-amino-6-(2-oxazolyl)purin-9-yl group as a base, wherein the 4- and/or 5-position of the thiazolyl or oxazolyl group may be substituted.

2. The nucleoside, nucleotide or derivative thereof according to claim 1, which has a 2-amino-6-(2-thiazolyl)purin-9-yl group as a base, wherein the 4- and/or 5-position of the thiazolyl group may be substituted.

3. The nucleoside, nucleotide or derivative thereof according to claim 1, wherein the 4- and/or 5-position of the thiazolyl or oxazolyl group is substituted with a lower alkyl group.

4. The nucleoside, nucleotide or derivative thereof according to claim 1, which has a 2-amino-6-(2-thiazolyl)purin-9-yl group, a 2-amino-6-(4-methyl-2-thiazolyl)purin-9-yl group or a 2-amino-6-(5-methyl-2-thiazolyl)purin-9-yl group as a base.

5. The nucleoside, nucleotide or derivative thereof according to claim 1, which is selected from the group consisting of the following:
i) 2-amino-6-(2-thiazolyl)-9-(2-deoxy-β-D-ribofuranosyl)purine;
ii) 2-amino-6-(2-thiazolyl)-9-(β-D-ribofuranosyl)purine;
iii) 2-amino-6-(2-thiazolyl)-9-(2-deoxy-β-D-ribofuranosyl)purine phosphate;
iv) 2-amino-6-(2-thiazolyl)-9-β-D-ribofuranosyl)purine phosphate;
v) 2-amino-6-(4-methyl-2-thiazolyl)-9-(2-deoxy-β-D-ribofuranosyl)purine;
vi) 2-amino-6-(4-methyl-2-thiazolyl)-9-(β-D-ribofuranosyl)purine;
vii) 2-amino-6-(4-methyl-2-thiazolyl)-9-(2-deoxy-β-D-ribofuranosyl)purine phosphate;
viii) 2-amino-6-(4-methyl-2-thiazolyl)-9-(β-D-ribofuranosyl)purine phosphate;
ix) 2-amino-6-(5-methyl-2-thiazolyl)-9-(2-deoxy-β-D-ribofuranosyl)purine;
x) 2-amino-6-(5-methyl-2-thiazolyl)-9-β-D-ribofuranosyl)purine;
xi) 2-amino-6-(5-methyl-2-thiazolyl)-9-(2-deoxy-β-D-ribofuranosyl)purine phosphate; and
xii) 2-amino-6-(5-methyl-2-thiazolyl)-9-β-D-ribofuranosyl)purine phosphate.

6. The nucleoside, nucleotide or derivative thereof according to any one of claims 1 to 5, which is in the form of a phosphoroamidite derivative.

7. A nucleic acid incorporating at least one nucleotide according to claim 1.

8. A nucleic acid incorporating at least one nucleotide according to claim 1, wherein said nucleotide forms a base pair with a nucleotide having a 5-substituted or unsubstituted-2-oxo(1H)-pyridin-3-yl group as a base.

9. The nucleic acid according to claim 7, which is a tRNA, mRNA, antisense DNA, antisense RNA, a ribozyme or an aptamer.

10. The nucleic acid according to claim 7, which encodes all or part of a protein or peptide.

11. A method for preparing a nucleic acid incorporating a nucleotide having a 5-substituted or unsubstituted-2-oxo(1H)-pyridin-3-yl group as a base, which comprises:
effecting transcription, replication or reverse transcription by using, as a template, a nucleic acid containing a nucleotide having a 2-amino-6-(2-thiazolyl)purin-9-yl group or a 2-amino-6-(2-oxazolyl)purin-9-yl group as a base, wherein the 4- and/or 5-position of the thiazolyl or oxazolyl group may be substituted, so that the nucleotide having a 5-substituted or unsubstituted-2-oxo(1H)-pyridin-3-yl group as a base is incorporated at a site complementary to the nucleotide having a 2-amino-6-(2-thiazolyl)purin-9-yl group or a 2-amino-6-(2-oxazolyl)purin-9-yl group as a base.

12. The method according to claim 11, wherein two or more nucleotides having a 2-amino-6-(2-thiazolyl)purin-9-yl group or a 2-amino-6-(2-oxazolyl)purin-9-yl group are located adjacent to each other in the template.

13. A kit for use in the method according to claim 11, which comprises:
a nucleic acid containing a nucleotide having a 2-amino-6-(2-thiazolyl)purin-9-yl group or a 2-amino-6-(2-oxazolyl)purin-9-yl group as a base, wherein the 4- and/or 5-position of the thiazolyl or oxazolyl group may be substituted; and
a nucleotide having a 5-substituted or unsubstituted-2-oxo(1H)-pyridin-3-yl group as a base.
